(19) United States
(12) Patent Application Publication
(10) Pub. No.: US 2023/0416219 A1
(43) Pub. Date: Dec. 28, 2023

(54) R-MDMA CRYSTAL FORMS

(71) Applicant: Mind Medicine, Inc., New York, NY (US)

(72) Inventors: Stephen E. SCHNEIDER, Raleigh, NC (US); Gillian MOORE, Sedgefield (GB); Julian Scott NORTHEN, Tyne and Wear (GB)

(21) Appl. No.: 18/325,655

(22) Filed: May 30, 2023

Related U.S. Application Data
(60) Provisional application No. 63/355,576, filed on Jun. 25, 2022.

(51) Int. Cl. C07D 317/50

(52) U.S. Cl. CPC C07D 317/50; C07B 2200/13

(57) ABSTRACT

A composition of a crystalline form salt or polymorph of R-MDMA. A pharmaceutical composition of a crystalline form salt or polymorph of R-MDMA and pharmaceutically acceptable excipients. A method of treating an individual for a medical condition, by administering an effective amount of a composition of a crystalline form salt or polymorph of R-MDMA to the individual and treating the individual.

### BACKGROUND OF THE INVENTION

### 1. Technical Field

The present invention relates to compositions and methods for making crystal forms of R-MDMA.

### 2. Background Art

3,4-Methylenedioxymethamphetamine (MDMA) is a psychoactive drug that alters mood and perception, and is investigated as an adjunct in psychotherapy for posttraumatic stress disorder (PTSD), social anxiety, autism (Danforth, 2016; Danforth et al., 2018; Danforth et al., 2016; Mithoefer et al., 2019; Mithoefer et al., 2010; Oehen et al., 2013), and may later also be studied and used for a range of other medical conditions. Such conditions where MDMA or related substances may be useful include, but are not limited to, substance-use disorder, depression, anxiety disorder (including social anxiety), anxiety with life-threatening disease, personality disorder including narcistic and antisocial disorder, autism and other developmental disorders and obsessive-compulsive disorder. MDMA or related substances can also be used to enhance individual or couple therapy.

There are several side effects and safety concerns regarding MDMA. Abuse of MDMA can produce hyperpyrexia, neurocognitive defects, and increased rates of depression. MDMA can also be neurotoxic which limits its ability to be used chronically with repeat administration. Use of MDMA often impairs declarative memory, prospective memory, and higher cognitive skills. Neurocognitive deficits are associated with reduced serotonin transporter (SERT) in the hippocampus, parietal cortex, and prefrontal cortex. EEG and ERP studies have shown localized reductions in brain activity during neurocognitive performance. Deficits in sleep, mood, vision, pain, psychomotor skill, tremor, neurohormonal activity, and psychiatric status, have also been demonstrated. These effects are seen more with higher doses or longer use. (Parrott, Neuroscience & Biobehavioral Reviews, Volume 37, Issue 8, 2013, Pages 1466-1484).

MDMA has two enantiomers, S(+)-MDMA and R(−)-MDMA. The R enantiomer is thought to be more active (Nichols, et al. J. Med. Chem. 1986, 29, 2009-2015). It is believed that the neurotoxicity of racemic MDMA is caused by the S(+) enantiomer, not the R(−) enantiomer due to the low efficacy of the R(−) enantiomer as a releaser of dopamine. The R(−) enantiomer also does not produce hyperthermia. The R(−) enantiomer may have a lower risk of abuse. (Pitts, et al. Psychopharmacology (2018) 235:377-392). It has been shown that the enantiomers have different effects. R-MDMA and S-MDMA were evaluated for their effects in a parkinsonian animal model (Huot, et al., The Journal of Neuroscience, 2011, 31 (19):7190-7198), and it was found that R- MDMA, which is a selective compound for 5-HT2A receptors, decreased severity of peak-dose dyskinesia and increased duration of good ON-time, S-MDMA, which exhibits high affinity for SERT and moderate affinity for DAT, extended total duration of ON-time but exacerbated dyskinesia. This showed that racemic MDMA exerts simultaneous effects, reducing dyskinesia and extending ON-time, by 5-HT2A antagonism and SERT-selective mixed monoamine uptake inhibition, which arise from its R and S enantiomers, respectively. Therefore, it can be advantageous to use R-MDMA in treatments.

R-MDMA free base is an oil. Stabilization as a crystalline salt is needed to facilitate handling, enable long term storage, and drug product manufacture. R-MDMA HCl salt (CAS 69558-31-2) has been reported in the literature (S. Llabrés et al. European Journal of Medicinal Chemistry 81 (2014) 35-46, The Journal of Neuroscience, May 11, 2011, 31 (19):7190 —7198, J. Med. Chem. 1986, 29, 2009-2015). However, these preparations of R-MDMA HCl provided no or few details and/or are not suitable for large scale manufacture. The solid-state properties also have not been reported.

Therefore, there remains a need for compositions of R-MDMA that can be produced on an appropriate scale for use in treatments.

### SUMMARY OF THE INVENTION

The present invention provides for a composition of a crystalline form salt or polymorph of R-MDMA.

The present invention provides for a pharmaceutical composition of a crystalline form salt or polymorph of R-MDMA and pharmaceutically acceptable excipients.

The present invention provides for a method of treating an individual for a medical condition, by administering an effective amount of a composition of a crystalline form salt or polymorph of R-MDMA and treating the individual.

### DESCRIPTION OF THE DRAWINGS

Other advantages of the present invention are readily appreciated as the same becomes better understood by reference to the following detailed description when considered in connection with the accompanying drawings wherein:

FIG. 1 is an XRPD Diffractogram of R-MDMA HCl Pattern A;

FIG. 2 is a ¹H NMR spectrum of R-MDMA HCl Pattern A;

FIG. 3 is a DSC and TGA thermograph of R-MDMA HCl Pattern A;

FIG. 4 is a DVS profile of R-MDMA HCl Pattern A;

FIG. 5 is XRPD Diffractograms of R-MDMA HCl Pattern A at ambient conditions (middle) and at 0% relative humidity (top) and 90% relative humidity (bottom);

FIG. 6A is an XRPD Diffractogram of R-MDMA HBr Pattern A, FIG. 6B is a ¹H NMR spectrum of R-MDMA HBr Pattern A, and FIG. 6C is a DSC and TGA thermograph of R-MDMA HBr Pattern A;

FIG. 7 is a DVS profile of R-MDMA HBr Pattern A;

FIG. 8 is XRPD Diffractograms of R-MDMA HBr Pattern A at ambient conditions (bottom) and at 0% relative humidity (top) and 90% relative humidity (middle);

FIG. 9A is an XRPD Diffractogram of R-MDMA Phosphate Pattern C, FIG. 9B is a ¹H NMR spectrum of R-MDMA Phosphate Pattern C, and FIG. 9C is a DSC and TGA thermograph of R-MDMA Phosphate Pattern C;

FIG. 10A is a DVS profile of R-MDMA Phosphate Pattern C, and FIG. 10B is XRPD Diffractograms of R-MDMA Phosphate Pattern C at ambient conditions (middle) and at 0% relative humidity (bottom) and 90% relative humidity (top);

FIG. 11A is an XRPD Diffractogram of R-MDMA D-Tartrate Pattern C, FIG. 11B is a ¹H NMR spectrum of R-MDMA D-Tartrate Pattern C, and FIG. 11C a DSC and TGA thermograph of R-MDMA D-Tartrate Pattern C;

FIG. 12A is a DVS profile of R-MDMA Tartrate D-Pattern C, and FIG. 12B is XRPD Diffractograms of R-MDMA D-Tartrate Pattern C at ambient conditions (middle) and at 0% relative humidity (top) and 90% relative humidity (bottom);

FIG. 13A is an XRPD Diffractogram of R-MDMA hemi fumarate Pattern A, FIG. 13B is a ¹H NMR spectrum of R-MDMA hemi fumarate Pattern A, and FIG. 13C is a DSC and TGA thermograph of R-MDMA hemi fumarate Pattern A;

FIG. 14A is a DVS profile of R-MDMA hemi fumarate Pattern A, and FIG. 14B is an overlay of XRPD Diffractograms of R-MDMA hemi fumarate Pattern A at ambient conditions (middle) and at 0% relative humidity (bottom) and 90% relative humidity (top);

FIG. 15 is an XRPD Diffractogram of R-MDMA hemi oxalate Pattern A/A′;

FIG. 16 is a ¹H NMR spectrum of R-MDMA hemi oxalate Pattern A/A′;

FIG. 17 a DSC and TGA thermograph of R-MDMA hemi oxalate Pattern A/A′;

FIG. 18 is a DVS profile of R-MDMA hemi oxalate Pattern A/A′;

FIG. 19 is an overlay of XRPD Diffractograms of R-MDMA hemi oxalate Pattern A/A′ at ambient conditions (middle) and at 0% relative humidity (top) and 90% relative humidity (bottom);

FIGS. 20A-20D are optical micrographs of R-MDMA HCl Pattern A, FIG. 20A is at 4× without oil, FIG. 20B is at 10× objective without oil, FIG. 20C is at 4× objective with oil, and FIG. 20D is at 10× objective with oil;

FIG. 21 is a representation of the asymmetric unit of the R-MDMA hydrochloride structure as determined by single crystal x-ray diffraction;

FIG. 22 is a representation of the crystal packing of the R-MDMA hydrochloride as determined by single crystal x-ray diffraction;

FIG. 23 is an overlay of the XRPD Diffractograms of R-MDMA maleate isolated from IPA (top, low crystallinity), an attempted hemi-salt from ethanol (middle, Pattern A), and a mono salt isolated from THF (bottom, Pattern A);

FIG. 24 is an overlay of the XRPD Diffractograms of R-MDMA maleate Pattern A isolated from THF (top, lower crystallinity), IPA (middle), and DCM (bottom);

FIG. 25 is an overlay of the XRPD Diffractograms of R-MDMA hemi-meso tartrate isolated from THF (top, mix of Patterns A and C), DCM (middle, Pattern A), and THF (bottom, Pattern B);

FIG. 26 is an XRPD Diffractogram of R-MDMA citrate;

FIG. 27 is an overlay of the XRPD Diffractograms of R-MDMA phosphate isolated from THF (top, Pattern C), IPA (middle, Pattern A), and DCM (bottom, Pattern B),

FIG. 28 is an overlay of the XRPD Diffractograms of R-MDMA hemi-naphthylene-1,5-disulphonate isolated from THF (top), IPA (middle), and DCM (bottom);

FIG. 29 is an overlay of the XRPD Diffractograms of R-MDMA sulfate Pattern B (top) and Pattern A (bottom) both isolated from DCM;

FIG. 30 is an overlay of the XRPD Diffractograms of R-MDMA mesylate isolated from THF (top) and DCM (bottom);

FIG. 31 is an overlay of the XRPD Diffractograms of R-MDMA acetate isolated from THF (top) and DCM (bottom);

FIG. 32 is an overlay of the XRPD Diffractograms of R-MDMA oxalate isolated from IPA (top), THF (middle), and DCM (bottom);

FIG. 33 is an XPRD Diffractogram of R-MDMA HBr pattern B;

FIG. 34 is an XPRD Diffractogram of R-MDMA phosphate pattern A;

FIG. 35 is an XPRD Diffractogram of R-MDMA phosphate pattern B;

FIG. 36 is an XPRD Diffractogram of R-MDMA tartrate pattern A;

FIG. 37 is an XPRD Diffractogram of R-MDMA tartrate pattern B;

FIG. 38 is an XPRD Diffractogram of R-MDMA maleate pattern A;

FIG. 39 is an XPRD Diffractogram of R-MDMA L-maleate pattern A;

FIG. 40 is an XPRD Diffractogram of R-MDMA hemi-napthylene-1,5-disulfonate pattern A;

FIG. 41 is an XPRD Diffractogram of R-MDMA hemi-fumarate pattern A;

FIG. 42 is an XPRD Diffractogram of R-MDMA oxalate pattern A;

FIG. 43 is an XPRD Diffractogram of R-MDMA sulfate pattern A;

FIG. 44 is an XPRD Diffractogram of R-MDMA sulfate pattern B;

FIG. 45 is an XPRD Diffractogram of R-MDMA mesylate pattern A; and

FIG. 46 is an XPRD Diffractogram of R-MDMA acetate pattern A.

### DETAILED DESCRIPTION OF THE INVENTION

The present invention provides for salts and polymorphs of R-MDMA, which can be used to prepare a stable crystalline form of R-MDMA for an appropriate scale for manufacture and to use in treatments.

The salt can be, but is not limited to, hydrochloride (HCl), hydrobromide (HBr), maleate, L-malate, D-tartrate, hemi-meso-tartrate, hemi-L-tartrate, citrate, phosphate, hemi-naphthylene-1,5-disulphonate, hemi-fumarate, sulfate, mesylate, acetate, hemi-oxalate, or oxalate. More specifically, the salt can be in a particular pattern such as, but not limited to, hydrochloride pattern A, phosphate pattern A, phosphate pattern B, phosphate pattern C, HBr pattern A, HBr pattern B, HBr pattern C, hemi-L-tartrate pattern A, hemi-meso-tartrate pattern B, hemi-meso-tartrate pattern C, meso-tartrate pattern A, meso-tartrate pattern B, sulfate pattern A, sulfate pattern B, D-tartrate pattern A, D-tartrate pattern B, D-tartrate pattern C, D-tartrate pattern D, D-tartrate pattern E, L-maleate pattern A, maleate pattern A, maleate pattern B, hemi naptheylene-1,5-disulfonate pattern A, hemi naptheylene-1,5-disulfonate pattern B, hemi-oxalate pattern A, hemi-oxalate pattern A′, hemi-fumarate pattern A, hemi-fumarate pattern A′, mesylate pattern A, acetate pattern A, citrate pattern A, fumarate pattern A, or oxalate pattern A.

As further detailed below, when the acid is hydrochloric acid, the crystalline form can be characterized by an x-ray powder diffraction pattern having peaks expressed as 2θ at about 15.8, about 17.5, about 19.7, about 24.8, and about 24.9. When the acid is hydrobromic acid, the crystalline form pattern A can be characterized by an x-ray powder diffraction pattern having peaks expressed as 2θ at about 13.9, about 16.3, about 19.8, about 20.5, and about 24.0. When the acid is phosphoric acid, the crystalline form pattern C can be characterized by an x-ray powder diffraction pattern having peaks expressed as 2θ at about 13.4, about 14.6, about 17.4, about 18.7, and about 22.1. When the acid is D-tartaric acid, the crystalline form pattern C can be characterized by an x-ray powder diffraction pattern having peaks expressed as 2θ at about 6.0, about 12.0, about 13.3, about 17.9, and about 24.1. When the acid is fumaric acid, the crystalline form can be characterized by an x-ray powder diffraction pattern obtained by irradiation with Cu Kα x-rays having peaks expressed as 2θ at about 17.2, about 18.6, about 19.2, about 19.5, and about 21.8, and the salt can be a hemi-salt. When the acid is oxalic acid, the crystalline form can be characterized by an x-ray powder diffraction pattern obtained by irradiation with Cu Kα x-rays having peaks expressed as 2θ at about 15.2, about 16.4, about 16.8, about 19.3, and about 21.3, and the salt can be a hemi-salt.

When the acid is hydrobromic acid, the crystalline form pattern B can be characterized by an x-ray powder diffraction pattern obtained by irradiation with Cu Kα x-rays having peaks expressed as 2θ at about 13.9, about 16.2, about 16.9, about 20.5, and about 24.1. When the acid is phosphoric acid, the crystalline form pattern A can be characterized by an x-ray powder diffraction pattern having peaks expressed as 2θ at about 14.5, about 17.4, about 22.0, about 24.7, and about 24.9. When the acid is phosphoric acid, the crystalline form pattern B can be characterized by an x-ray powder diffraction pattern having peaks expressed as 2θ at about 12.9, about 13.8, about 17.1, about 26.8, and about 27.8. When the acid is D-tartaric acid, the crystalline form pattern A can be characterized by an x-ray powder diffraction pattern having peaks expressed as 2θ at about 5.6, about 11.3, about 15.4, about 17.2, and about 17.8. When the acid is D-tartaric acid, the crystalline form pattern B can be characterized by an x-ray powder diffraction pattern having peaks expressed as 2θ at about 5.1, about 16.3, about 19.3, about 20.4, and about 21.8. When the acid is maleic acid, the crystalline form can be characterized by an x-ray powder diffraction pattern having peaks expressed as 2θ at about 14.9, about 18.0, about 25.2, about 25.9, and about 27.9. When the acid is malic acid, the crystalline form can be characterized by an x-ray powder diffraction pattern having peaks expressed as 2θ at about 17.8, about 18.1, about 19.3, about 26.5, and about 27.3. When the acid is napthylene-1,5-disulfonic acid, the crystalline form can be characterized by an x-ray powder diffraction pattern having peaks expressed as 2θ at about 14.6, about 15.2, about 15.8, about 16.8, and about 22.9. The salt can also be a hem i-salt. When the acid is oxalic acid, the crystalline form can be characterized by an x-ray powder diffraction pattern having peaks expressed as 2θ at about 4.8, about 14.6, about 16.8, about 19.9, and about 21.0. When the acid is sulfuric acid, the crystalline form pattern A can be characterized by an x-ray powder diffraction pattern having peaks expressed as 2θ at about 14.9, about 17.8, about 21.0, about 21.2, and about 23.8. When the acid is sulfuric acid, the crystalline form pattern B can be characterized by an x-ray powder diffraction pattern having peaks expressed as 2θ at about 16.4, about 19.1, about 23.9, about 25.9, and about 27.8. When the acid is methanesulfonic acid, the crystalline form can be characterized by an x-ray powder diffraction pattern having peaks expressed as 2θ at about 16.2, about 17.9, about 18.5, about 21.2, and about 26.9. When the acid is acetic acid, the crystalline form can be characterized by an x-ray powder diffraction pattern having peaks expressed as 2θ at about 17.7, about 18.0, about 18.6, about 19.7, and about 20.3.

The salt or polymorph of R-MDMA can be administered in a dose of 10-1000 mg. MDMA is an agonist that primarily releases monoamines (serotonin, norepinephrine and dopamine) and possibly also oxytocin typically by interacting with the membrane monoamine transporters (serotonin, norepinephrine, or dopamine transporter) (Hysek et al., 2014; Hysek et al., 2012b; Simmler et al., 2013; Verrico et al., 2007).

The composition can also include prodrugs of salts or polymorphs of R-MDMA. A “prodrug” as used herein, refers to a compound that includes a moiety attached to an active drug substance that is metabolized after administration to an individual and the compound is converted into the active drug substance. Using a prodrug allows for improving how the active drug is absorbed, distributed, metabolized, and excreted. Prodrugs can be used to prevent release of the active drug in the gastrointestinal tract upon administration so that the drug can be released more favorably elsewhere in the body.

The prodrug compound includes a chemical modification to salt or polymorph of R-MDMA, such as an amino acid covalently attached thereto. The addition of the amino acid makes the active compound inactive mainly by preventing interaction with monoamine transporter, which is the site of action but also affecting bioavailability/rate of absorption. The amino acid can be lysine or any other amino acid such as alanine, arginine, asparagine, aspartic acid, cysteine, glutamine, glutamic acid, glycine, histidine, isoleucine, leucine, methionine, phenylalanine, proline, serine, threonine, tryptophan, tyrosine, or valine and typically attached to the amine (N)-group of R-MDMA and hence reducing pharmacological activity at the primary site of action (cell-membrane monoamine transporters including serotonin, dopamine and norepinephrine transporter), and also altering extent and rate of absorption and mainly releasing active substance in the circulation after absorption of the inactive compound. The amino acid can be any other natural or synthetic amino acid. Any other chemical modification can also be used.

Using a salt or polymorph of R-MDMA allows for daily use. The compositions are particularly useful in continual slow-release formulations, such as transdermal patches, that can provide a low dose over a long period of time. The compositions can also be administered in an intranasal spray. The composition can also be in a liquid dosage form such as, but not limited to, suspensions, solutions, emulsions, elixirs, tinctures, sprays, syrups, gels, magmas, liniments, lotions, ointments, pastes, drops, or inhalants. The composition can be in a solid dosage form such as, but not limited to, capsules, films, lozenge, patch, powder, tablets, pellets, pills, or troches.

The compound of the present invention is administered and dosed in accordance with good medical practice, considering the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including but not limited to more rapid recovery, or improvement or elimination of symptoms and other indicators as are selected as appropriate measures by those skilled in the art.

In the method of the present invention, the compound of the present invention can be administered in various ways. It should be noted that it can be administered as the compound and can be administered alone or as an active ingredient in combination with pharmaceutically acceptable carriers, diluents, adjuvants, and vehicles. The compounds can be administered orally, subcutaneously, or parenterally including sublingual, buccal, inhalation, intravenous, intramuscular, and intranasal administration. Implants of the compounds are also useful. The patient being treated is a warm-blooded animal and, in particular, mammals including man. The pharmaceutically acceptable carriers, diluents, adjuvants, and vehicles as well as implant carriers generally refer to inert, non-toxic solid or liquid fillers, diluents or encapsulating material not reacting with the active ingredients of the invention.

The doses can be single doses or multiple doses over a period of several days, weeks or months. The treatment generally has a length proportional to the length of the disease process and drug effectiveness and the patient species being treated.

When administering the compound of the present invention orally, it will generally be formulated in an immediate release capsule, immediate release tablet, modified release capsule or tablet (including enteric coatings), solution or suspension. When administering the compound of the present invention parenterally, it will generally be formulated in a sublingual or buccal orally dissolving tablet, dissolving film, intranasal powder, intranasal solution, inhaled powder, inhaled solution, transdermal patch, transdermal patch with microneedles or other permeation enhancers, or as a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

The present invention provides for a method of treating an individual for a medical disorder, by administering an effective amount of a composition of a salt or polymorph of R-MDMA to the individual, and treating the individual. The method can further include preventing or reducing side effects of neurotoxicity, hyperthermia and dependence/addiction experienced with racemic MDMA. Any of the prodrugs listed above can also be used.

Specifically, the compositions can be used in treating medical disorders or conditions including post-traumatic stress disorder, social anxiety, autism spectrum disorder, substance use disorder, depression, anxiety disorder, anxiety with life-threatening disease, personality disorder including narcistic or antisocial personality disorder, schizophrenia, obsessive compulsive disorder, couple therapy, enhancement of any psychotherapy by inducing feelings of well-being connectivity, trust, love, empathy, openness, and pro-sociality, and enhancing therapeutic bond in any psychotherapy of patients or neurotic/healthy subjects.

The invention is further described in detail by reference to the following experimental examples. These examples are provided for the purpose of illustration only and are not intended to be limiting unless otherwise specified. Thus, the invention should in no way be construed as being limited to the following examples, but rather, should be construed to encompass all variations which become evident as a result of the teaching provided herein.

### EXAMPLE 1

### General Procedure for the Preparation of Salts of R-MDMA

A salt screen was conducted using stock solutions of each acid prepared as indicated in Table 1. A stock solution of R-MDMA free base (1 g) in IPA (10 ml) was prepared at ambient temperature. Aliquots (0.4 ml, ˜30 mg) of the solution were charged to crystallization tubes. The solutions were heated to 50° C. and the relevant acid charged (1 equivalent) in one single aliquot. The solutions were equilibrated at 50° C. for 1 hour and then cooled to ambient temperature and equilibrated for 24 hours. Where suspensions were obtained, solids were isolated by filtration and dried in vacuo at 45° C. Where solutions persisted, further manipulation was required to obtain an isolable solid. The following methods were used primarily to induce crystallization and/or obtain a solid:

Reduction of solvent volume to ˜50% under a steady stream of nitrogen

Cooling to 0° C. and sub 0° C.

Addition of anti-solvent (MTBE) at ambient temperature followed by equilibration

Removal of solvent by a steady stream of nitrogen

Repeat scratching and trituration of resulting residue with MTBE followed by equilibration of solids where a suspension was obtained.

TABLE 1

| Acid | Solvents(s) | Molarity | Result |
|---|---|---|---|
| Hydrochloric | IPA, DCM, THF | 1M | Successful Salt Formation |
| Methane sulfonic | DCM, THF | 1M | Successful Salt Formation |
| Maleic | IPA, THF | 1M | Successful Salt Formation |
| (−)-(L)-Malic | IPA, DCM, THF | 1M | Successful Salt Formation |
| L-Tartaric | IPA, DCM, THF | 0.5M | Unsuccessful |
| D-Tartaric | IPA, DCM, THF | 0.5M | Successful Salt Formation |
| Meso-Tartaric | THF | 0.5M | Successful Salt Formation |
| Citric | IPA | 0.5M | Successful Salt Formation |
| Succinic | IPA, DCM, THF | 1M | Unsuccessful |
| Acetic | DCM, THF | 1M | Successful Salt Formation |
| p-Toluenesulfonic | IPA, DCM, THF | 1M | Unsuccessful |
| Sulfuric | DCM | 1M | Successful Salt Formation |
| Phosphoric | IPA, DCM, THF | 1M | Successful Salt Formation |
| Benzenesulfonic | IPA, THF | 1M | Unsuccessful |
| Xinafoic | IPA, DCM, THF | 0.5M | Unsuccessful |
| Hydrobromic | IPA, DCM, THF | 1M | Successful Salt Formation |
| Oxalic | IPA, DCM, THF | 1M | Successful Salt Formation |
| L-Aspartic | IPA, DCM, THF | Added as solid | Unsuccessful |
| Naphthylene- | IPA, DCM, THF | 1M | Successful Salt Formation |
| 1,5-disulfonic | | | |
| L-Glutamic | IPA, DCM, THF | Added as solid | Unsuccessful |
| Malonic | IPA, DCM, THF | 1M | Unsuccessful |
| Fumaric | IPA, DCM, THF | 0.25M | Successful Salt Formation |
| D-glucuronic | IPA, DCM, THF | Added as solid | Unsuccessful |
| Benzoic | IPA, DCM, THF | 1M | Unsuccessful |
| Gentisic | IPA, DCM, THF | 1M | Unsuccessful |

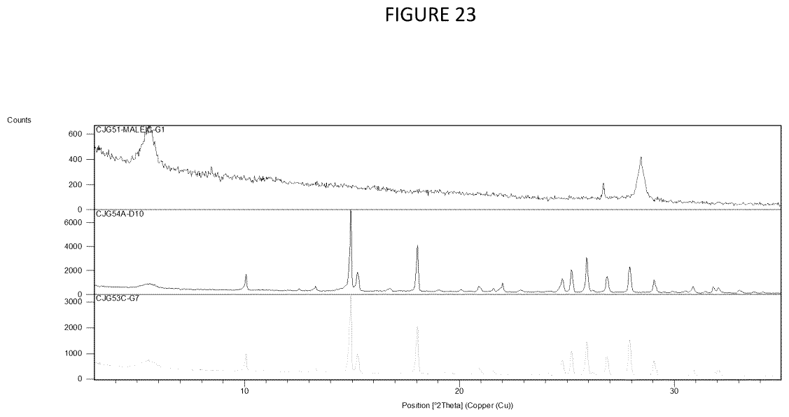

XRPD patterns for R-MDMA Maleate, R-MDMA L-Malate, R-MDMA Hemi Meso-tartrate, R-MDMA Citrate, R-MDMA Phosphate, R-MDMA Hemi Naphthylene-1,5-disulfonic, R-MDMA Sulfate, R-MDMA Mesylate, R-MDMA acetate, and R-MDMA Oxalate are shown in FIGS. 23-32.

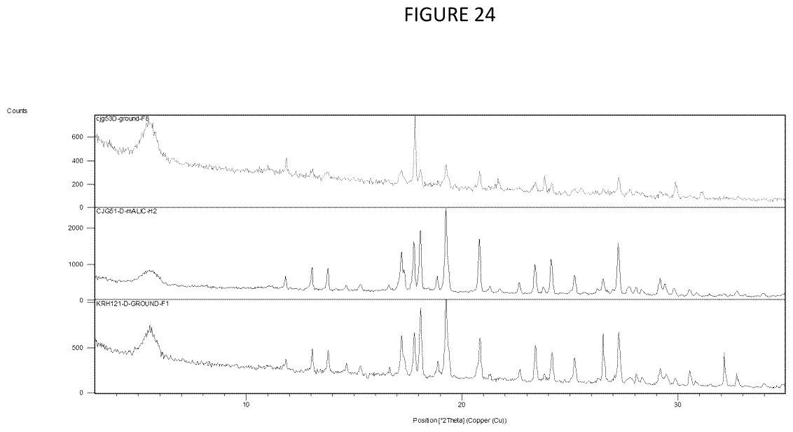

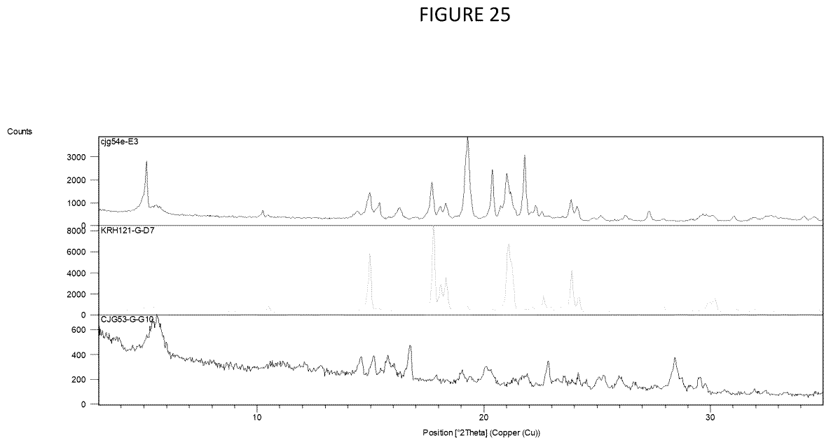

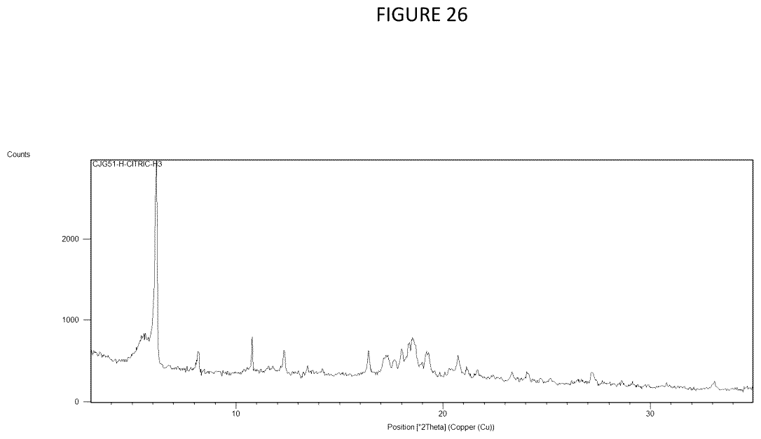

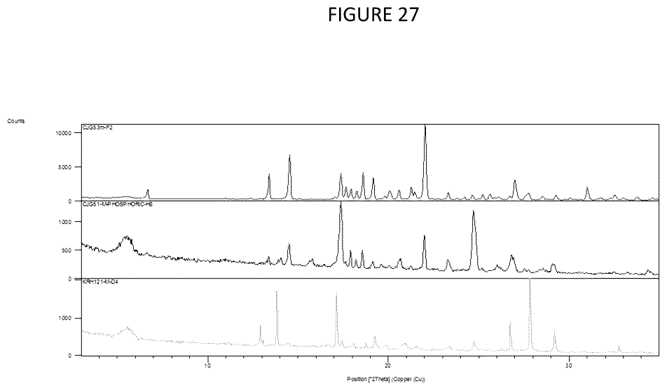

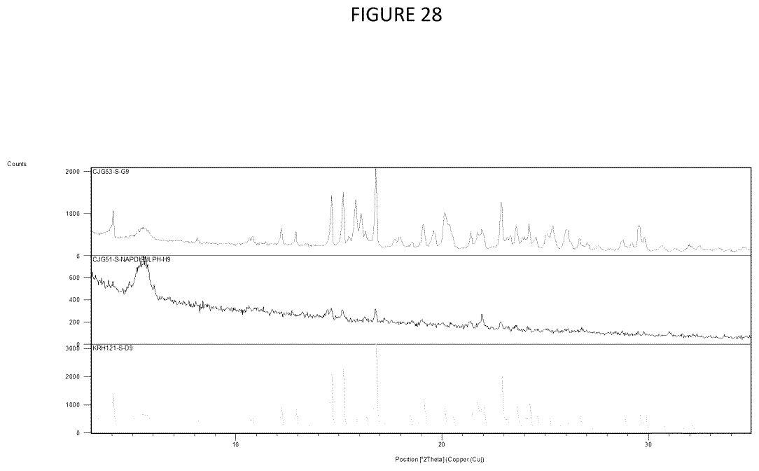

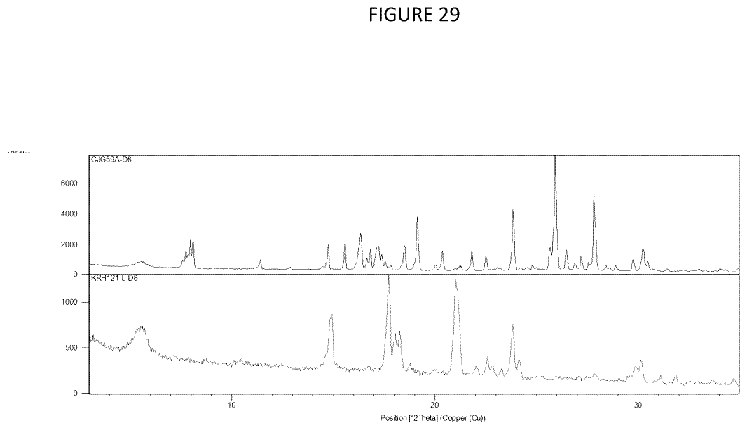

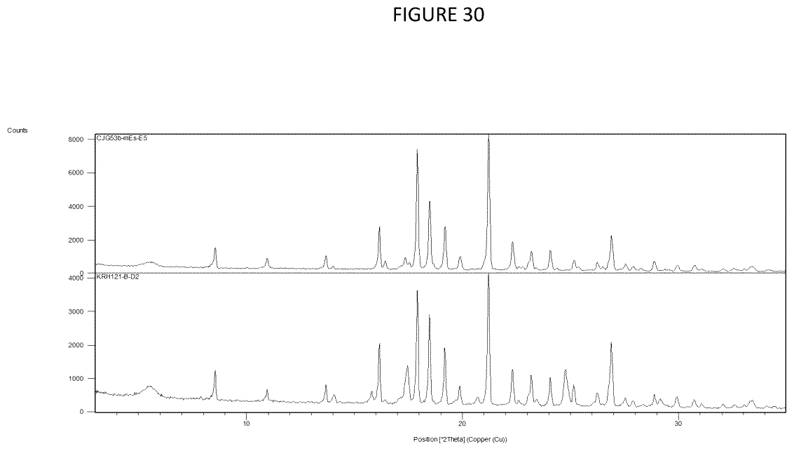

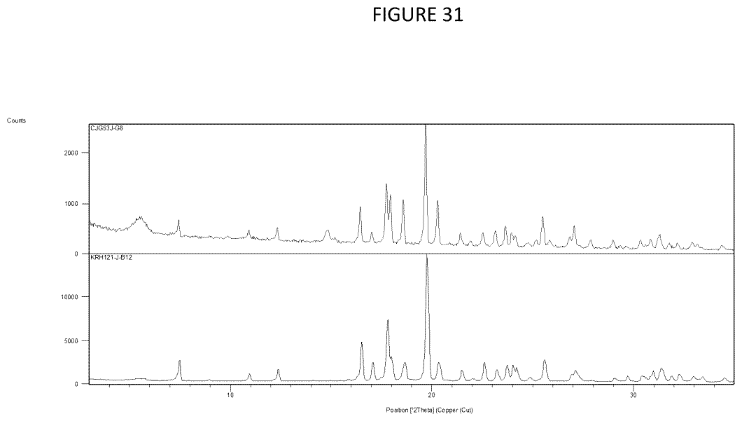

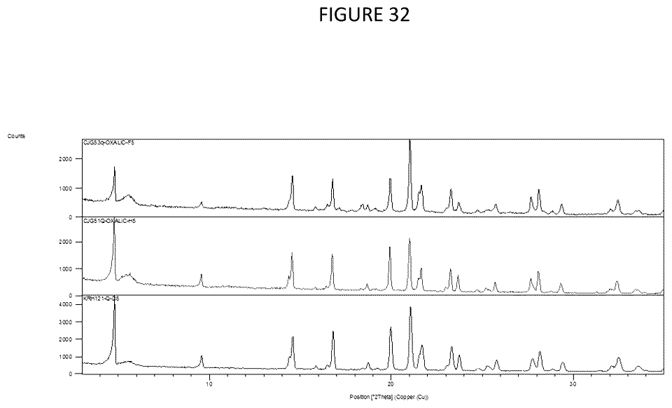

FIG. 23 shows an overlay of the XRPD Diffractograms of R-MDMA maleate isolated from IPA (top, low crystallinity), an attempted hemi-salt from ethanol (middle, Pattern A), and a mono salt isolated from THF (bottom, Pattern A). FIG. 24 shows an overlay of the XRPD Diffractograms of R-MDMA maleate Pattern A isolated from THF (top, lower crystallinity), IPA (middle), and DCM (bottom). FIG. 25 shows an overlay of the XRPD Diffractograms of R-MDMA hemi-meso tartrate isolated from THF (top, mix of Patterns A and C), DCM (middle, Pattern A), and THF (bottom, Pattern B). FIG. 26 shows an XRPD Diffractogram of R-MDMA citrate. FIG. 27 shows an overlay of the XRPD Diffractograms of R-MDMA phosphate isolated from THF (top, Pattern C), IPA (middle, Pattern A), and DCM (bottom, Pattern B). FIG. 28 shows an overlay of the XRPD Diffractograms of R-MDMA hemi-naphthylene-1,5-disulphonate isolated from THF (top), IPA (middle), and DCM (bottom). FIG. 29 shows an overlay of the XRPD Diffractograms of R-MDMA sulfate Pattern B (top) and Pattern A (bottom) both isolated from DCM. FIG. 30 shows an overlay of the XRPD Diffractograms of R-MDMA mesylate isolated from THF (top) and DCM (bottom). FIG. 31 shows an overlay of the XRPD Diffractograms of R-MDMA acetate isolated from THF (top) and DCM (bottom). FIG. 32 shows an overlay of the XRPD Diffractograms of R-MDMA oxalate isolated from IPA (top), THF (middle), and DCM (bottom).

### EXAMPLE 2

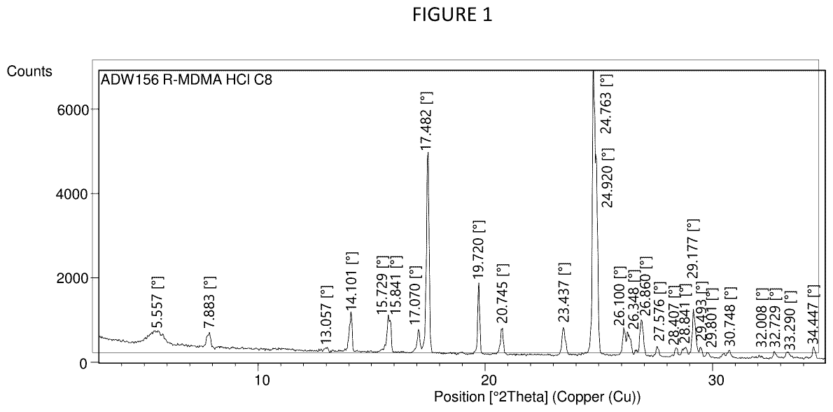

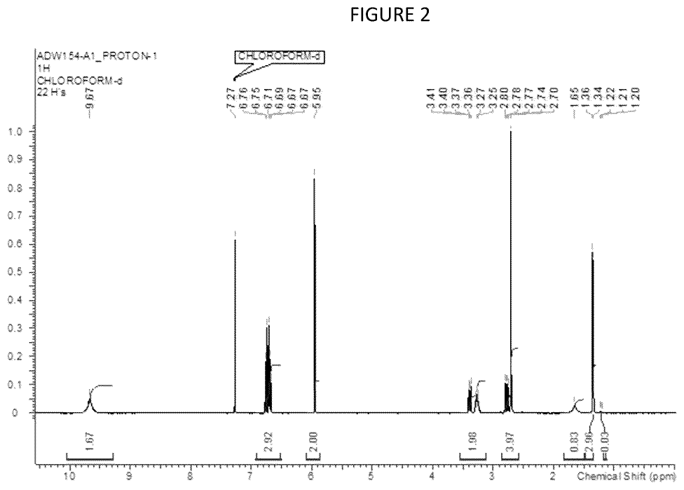

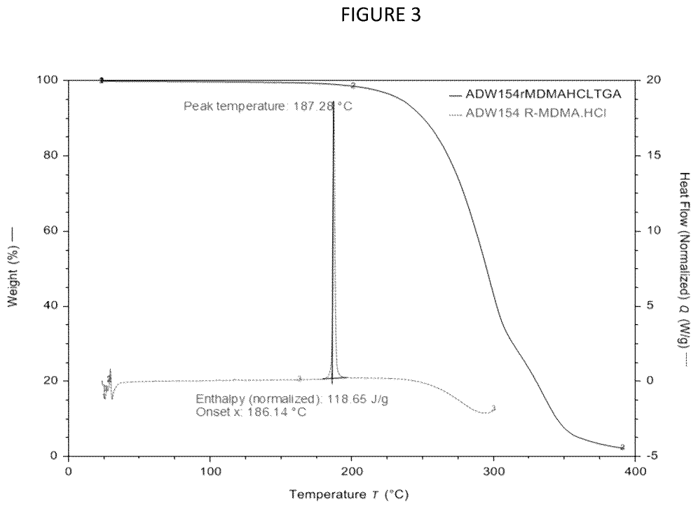

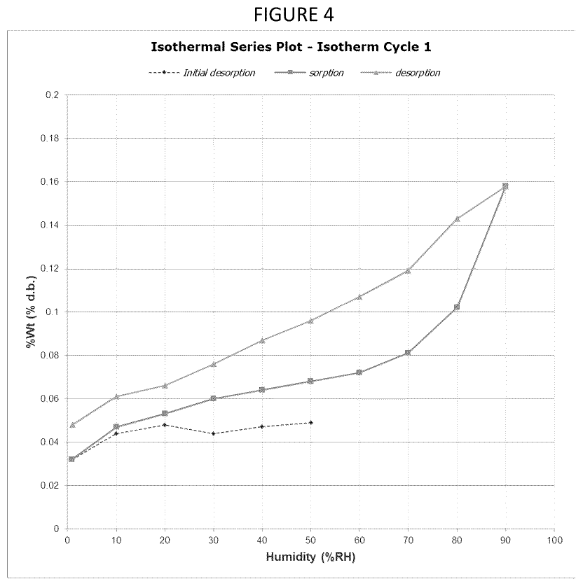

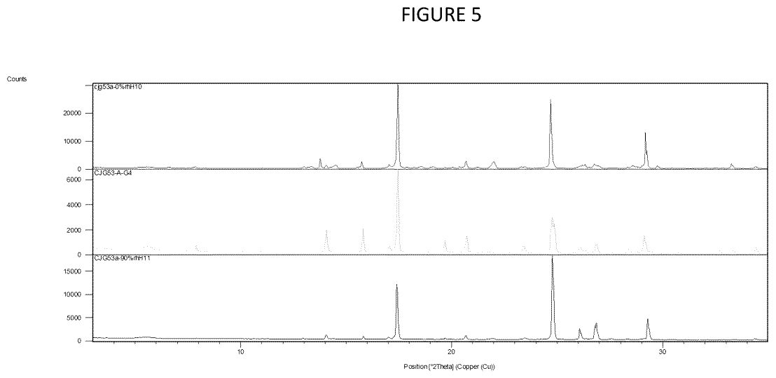

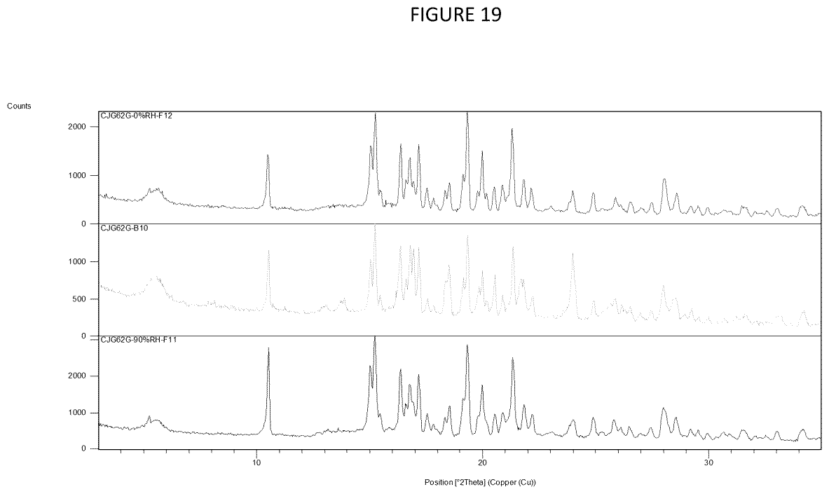

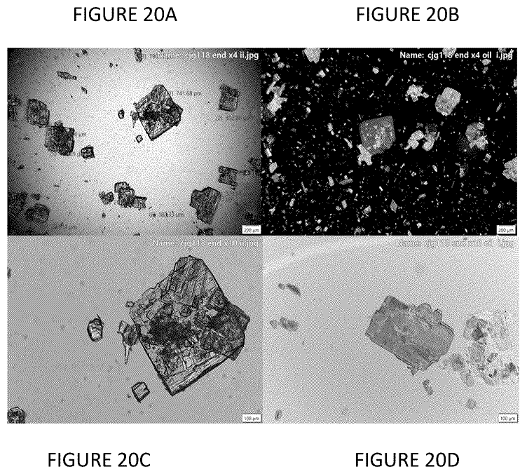

R-MDMA HCl salt pattern A was prepared. An XRPD pattern is shown in FIG. 1. A ¹H NMR spectrum is shown in FIG. 2. A combined DSC/TGA thermograph is shown in FIG. 3. FIG. 4 shows a DVS profile and FIG. 5 shows XRPD patterns at ambient conditions, 0% relative humidity, and 90% relative humidity. TABLE 2 shows a XRPD peak list. Optical micrographs of R-MDMA HCl Pattern A are shown in FIGS. 20A-20D.

TABLE 2

| Pos. [°2Theta] | Height [counts] | Rel. Int. [%] |
|---|---|---|
| 5.5572* | 291.82 | 4.27 |
| 7.8827 | 303.15 | 4.44 |
| 13.0575 | 86.93 | 1.27 |
| 14.1008 | 946.68 | 13.86 |
| 15.7291 | 860.20 | 12.59 |
| 15.8412 | 759.13 | 11.11 |
| 17.0695 | 560.49 | 8.20 |
| 17.4818 | 4848.55 | 70.97 |
| 19.7197 | 1722.78 | 25.22 |
| 20.7449 | 597.90 | 8.75 |
| 23.4371 | 657.45 | 9.62 |
| 24.7634 | 6831.40 | 100.00 |
| 24.9204 | 3673.06 | 53.77 |
| 26.0998 | 683.36 | 10.00 |
| 26.3478 | 467.14 | 6.84 |
| 26.8603 | 805.20 | 11.79 |
| 27.5765 | 227.72 | 3.33 |
| 28.4073 | 222.71 | 3.26 |
| 28.8410 | 216.13 | 3.16 |
| 29.1771 | 1023.13 | 14.98 |
| 29.4932 | 207.05 | 3.03 |
| 29.8012 | 106.04 | 1.55 |
| 30.7479 | 171.63 | 2.51 |
| 32.0077 | 28.83 | 0.42 |
| 32.7288 | 166.61 | 2.44 |
| 33.2899 | 149.29 | 2.19 |
| 34.4468 | 285.79 | 4.18 |

*The peak at 5.5572°2Theta is due the Kapton film used in the analysis and not related to R-MDMA HCl salt pattern A

### EXAMPLE 3

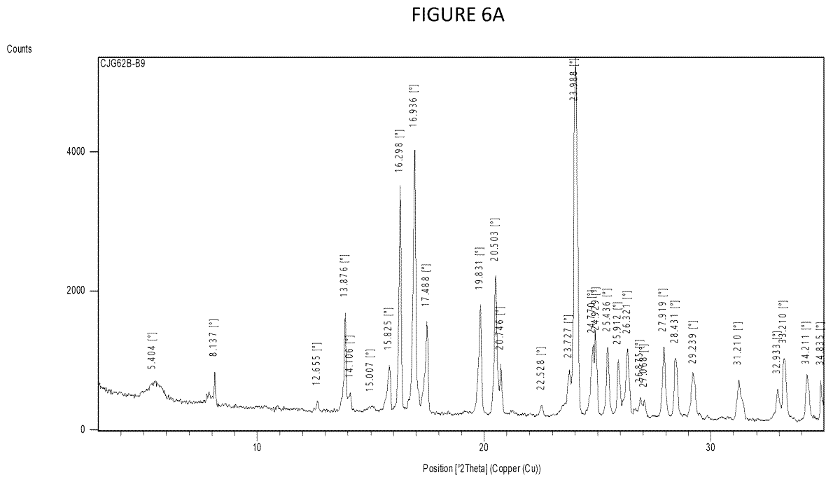

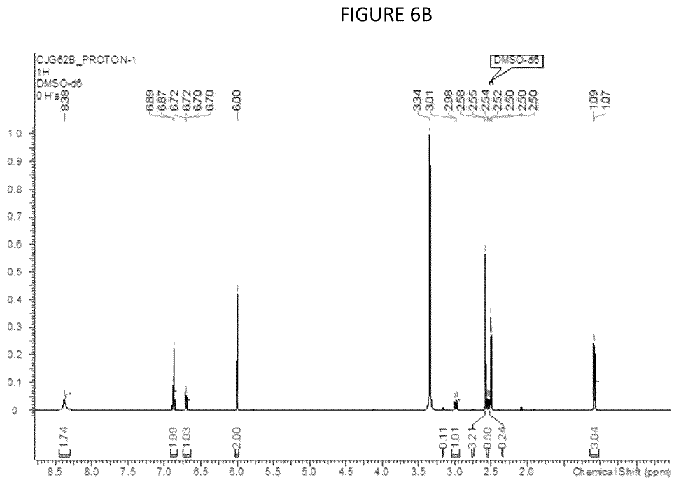

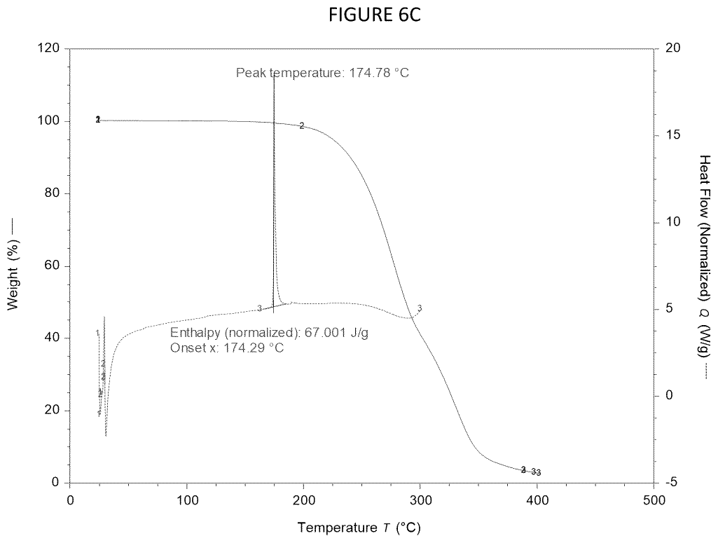

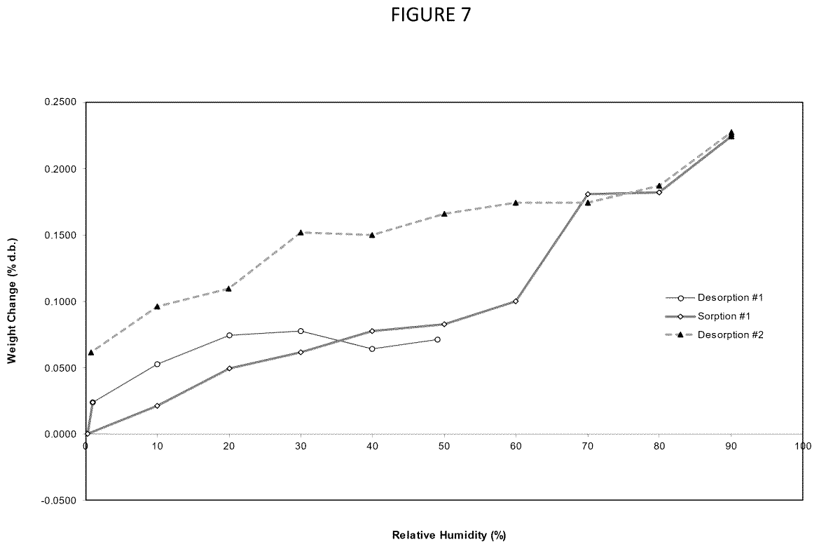

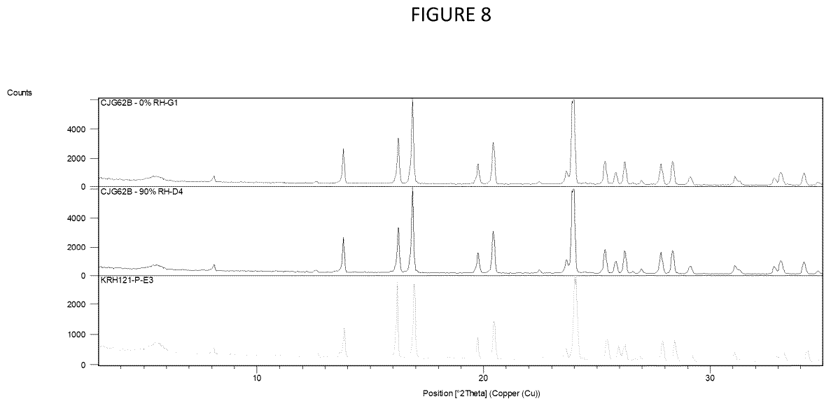

R-MDMA HBr salt pattern A was prepared. An XRPD pattern is shown in FIG. 6A. A ¹H NMR spectrum is shown in FIG. 6B. A combined DSC/TGA thermograph is shown in FIG. 6C. FIG. 7 shows a DVS profile and FIG. 8 shows XRPD patterns at ambient conditions, 0% relative humidity, and 90% relative humidity. TABLE 3 shows a peak list.

TABLE 3

| Pos. [°2Theta] | Height [counts] | Rel. Int. [%] |
|---|---|---|
| 5.4037* | 193.12 | 4.10 |
| 8.1368 | 478.42 | 10.16 |
| 12.6551 | 138.47 | 2.94 |
| 13.8761 | 1419.09 | 30.13 |
| 14.1061 | 265.63 | 5.64 |
| 15.0068 | 72.04 | 1.53 |
| 15.8255 | 673.12 | 14.29 |
| 16.2976 | 3244.46 | 68.89 |
| 16.9357 | 3894.80 | 82.70 |
| 17.4881 | 1321.50 | 28.06 |
| 19.8312 | 1577.27 | 33.49 |
| 20.5034 | 2006.71 | 42.61 |
| 20.7460 | 721.21 | 15.31 |
| 22.5278 | 159.84 | 3.39 |
| 23.7268 | 624.79 | 13.27 |
| 23.9884 | 4709.46 | 100.00 |
| 24.7697 | 987.25 | 20.96 |
| 24.9285 | 1036.96 | 22.02 |
| 25.4358 | 1019.65 | 21.65 |
| 25.9121 | 831.36 | 17.65 |
| 26.3210 | 992.66 | 21.08 |
| 26.8753 | 287.39 | 6.10 |
| 27.0681 | 227.78 | 4.84 |
| 27.9188 | 1034.56 | 21.97 |
| 28.4310 | 867.91 | 18.43 |
| 29.2394 | 577.73 | 12.27 |
| 31.2105 | 567.68 | 12.05 |
| 32.9325 | 447.82 | 9.51 |
| 33.2103 | 886.35 | 18.82 |
| 34.2108 | 582.02 | 12.36 |
| 34.8349 | 570.16 | 12.11 |

*The peak at 5.4037°2Theta is due the Kapton film used in the analysis and not related to R-MDMA HBr salt Pattern A

### EXAMPLE 4

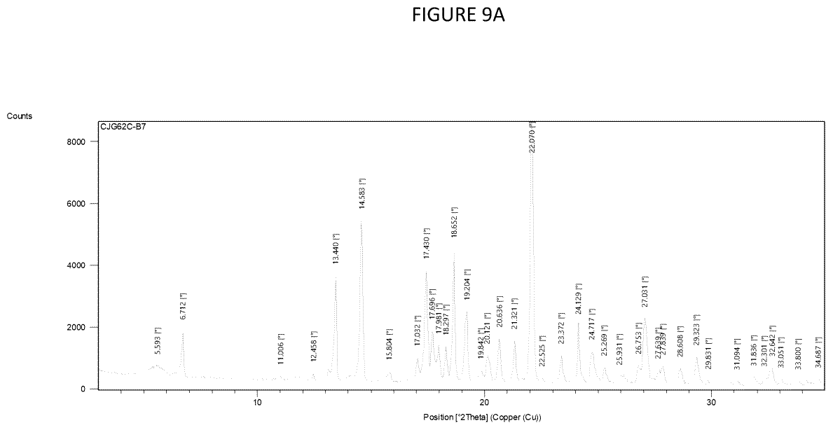

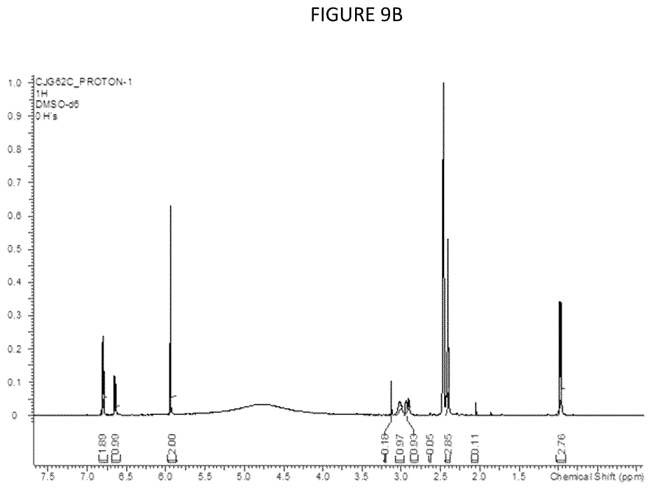

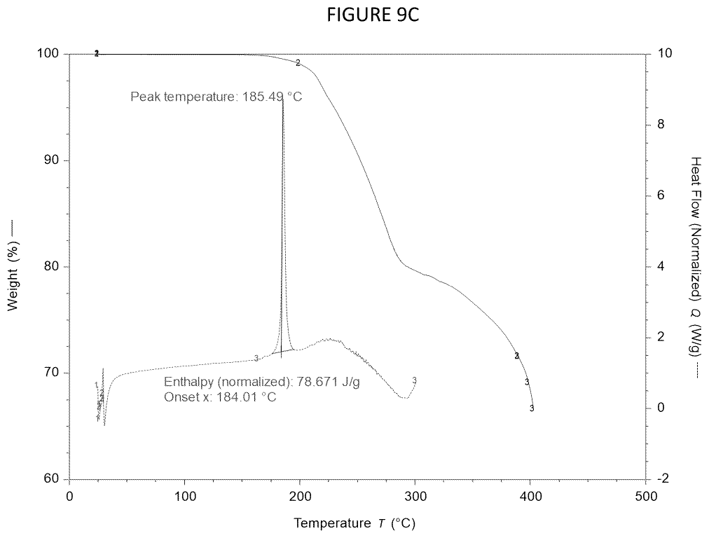

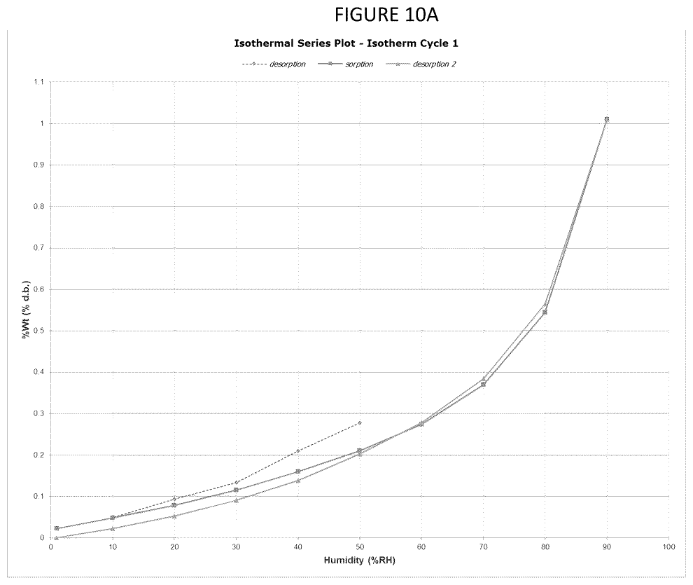

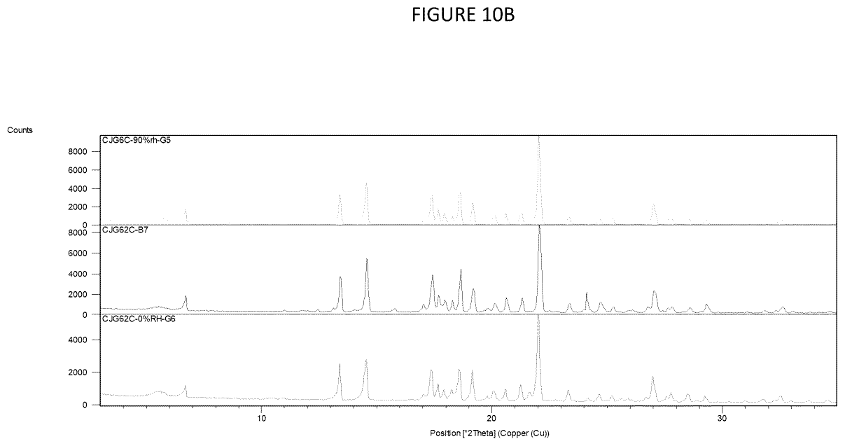

R-MDMA Phosphate salt pattern C was prepared. An XRPD pattern is shown in FIG. 9A. A ¹H NMR spectrum is shown in FIG. 9B. A combined DSC/TGA thermograph is shown in FIG. 9C. FIG. 10A shows a DVS profile and FIG. 10B shows XRPD patterns at ambient conditions, 0% relative humidity, and 90% relative humidity. TABLE 4 shows a peak list.

TABLE 4

| Pos. [°2Theta] | Height [counts] | Rel. Int. [%] |
|---|---|---|
| 5.5933* | 314.26 | 3.71 |
| 6.7120 | 1463.62 | 17.29 |
| 11.0061 | 117.13 | 1.38 |
| 12.4581 | 214.70 | 2.54 |
| 13.4401 | 3376.34 | 39.89 |
| 14.5831 | 5178.19 | 61.18 |
| 15.8037 | 309.37 | 3.66 |
| 17.0318 | 752.37 | 8.89 |
| 17.4296 | 3585.24 | 42.36 |
| 17.6959 | 1623.41 | 19.18 |
| 17.9810 | 1155.77 | 13.66 |
| 18.2968 | 1122.82 | 13.27 |
| 18.6521 | 4274.66 | 50.50 |
| 19.2043 | 2262.49 | 26.73 |
| 19.8418 | 357.63 | 4.23 |
| 20.1207 | 858.15 | 10.14 |
| 20.6363 | 1402.11 | 16.57 |
| 21.3206 | 1336.33 | 15.79 |
| 22.0698 | 8463.94 | 100.00 |
| 22.5246 | 149.77 | 1.77 |
| 23.3717 | 880.78 | 10.41 |
| 24.1294 | 1822.51 | 21.53 |
| 24.7174 | 1023.53 | 12.09 |
| 25.2689 | 507.17 | 5.99 |
| 25.9310 | 219.42 | 2.59 |
| 26.7534 | 571.42 | 6.75 |
| 27.0313 | 2091.66 | 24.71 |
| 27.6391 | 430.88 | 5.09 |
| 27.8387 | 556.84 | 6.58 |
| 28.6081 | 494.50 | 5.84 |
| 29.3231 | 872.29 | 10.31 |
| 29.8311 | 107.90 | 1.27 |
| 31.0937 | 104.60 | 1.24 |
| 31.8357 | 229.89 | 2.72 |
| 32.3008 | 213.60 | 2.52 |
| 32.6420 | 534.16 | 6.31 |
| 33.0506 | 164.93 | 1.95 |
| 33.8005 | 112.81 | 1.33 |
| 34.6870 | 176.75 | 2.09 |

*The peak at 5.5933°2Theta is due the Kapton film used in the analysis and not related to R-MDMA Phosphate salt Pattern C

### EXAMPLE 5

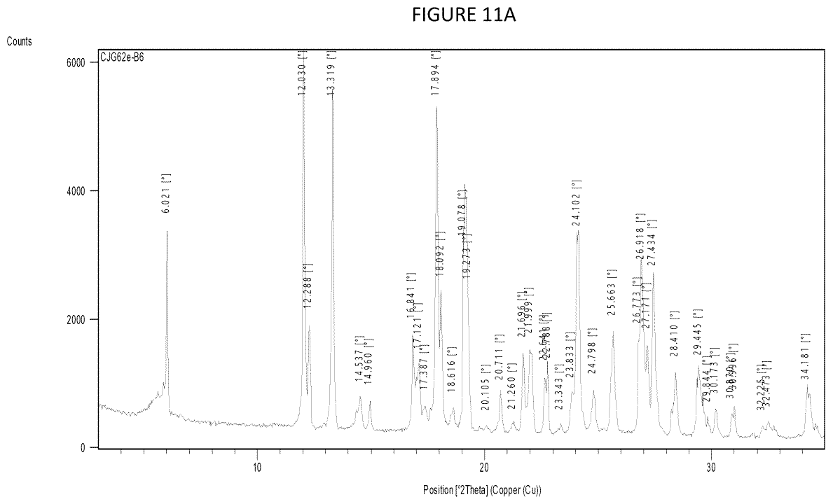

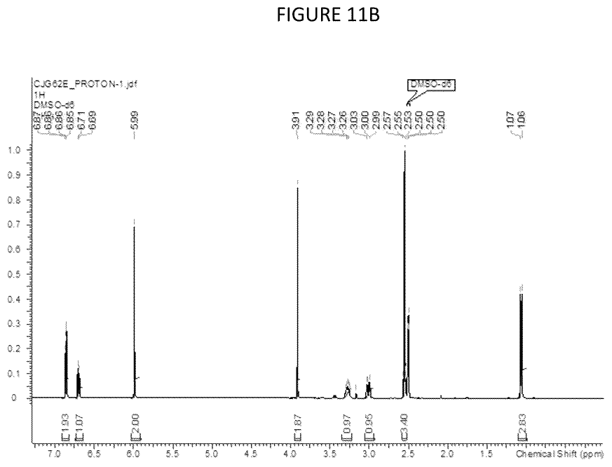

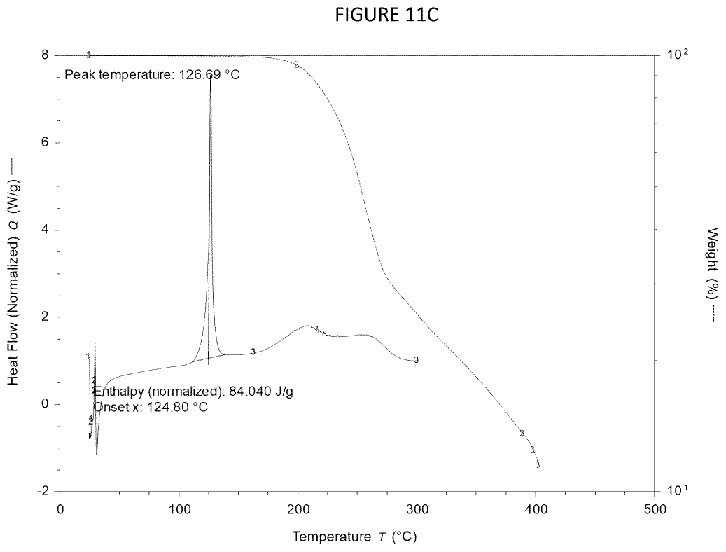

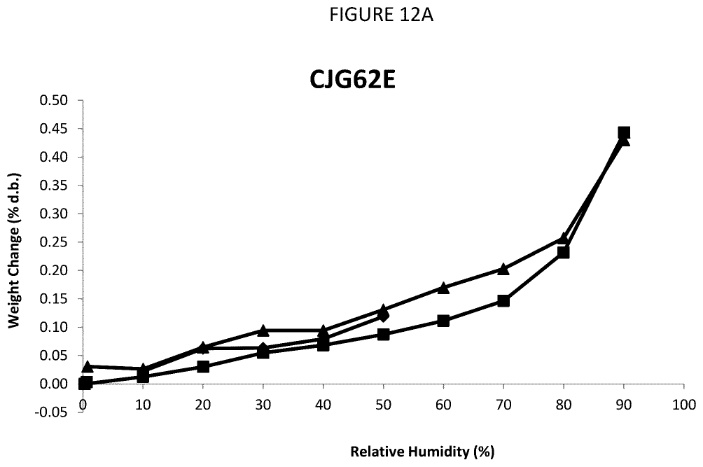

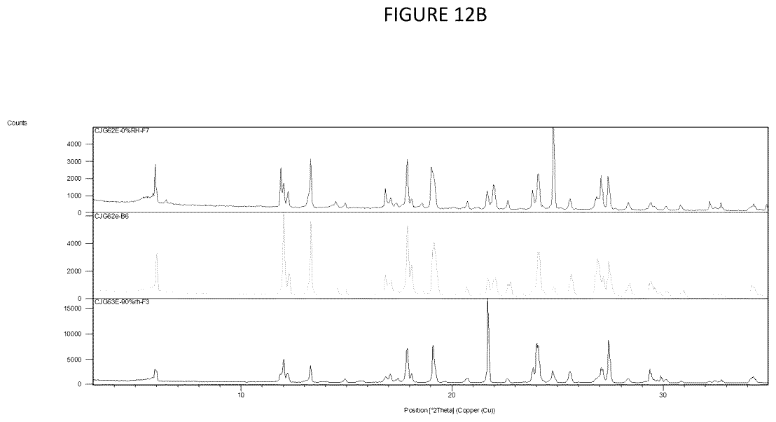

R-MDMA D-Tartrate salt pattern C was prepared. An XRPD pattern is shown in FIG. 11A. A ¹H NMR spectrum is shown in FIG. 11B. A combined DSC/TGA thermograph is shown in FIG. 11C. FIG. 12A shows a DVS profile and FIG. 12B shows XRPD patterns at ambient conditions, 0% relative humidity, and 90% relative humidity. TABLE 5 shows a peak list.

TABLE 5

| Pos. [°2Theta] | Height [counts] | Rel. Int. [%] |
|---|---|---|
| 6.0212 | 2938.98 | 49.89 |
| 12.0299 | 5890.44 | 100.00 |
| 12.2883 | 1592.45 | 27.03 |
| 13.3191 | 5495.43 | 93.29 |
| 14.5373 | 498.48 | 8.46 |
| 14.9605 | 455.50 | 7.73 |
| 16.8415 | 1500.75 | 25.48 |
| 17.1210 | 1034.74 | 17.57 |
| 17.3873 | 382.61 | 6.50 |
| 17.8942 | 5184.48 | 88.02 |
| 18.0921 | 2151.93 | 36.53 |
| 18.6161 | 358.40 | 6.08 |
| 19.0778 | 2800.58 | 47.54 |
| 19.2726 | 2128.07 | 36.13 |
| 20.1050 | 87.26 | 1.48 |
| 20.7111 | 568.66 | 9.65 |
| 21.2597 | 123.10 | 2.09 |
| 21.6960 | 1240.97 | 21.07 |
| 21.9990 | 1304.87 | 22.15 |
| 22.6411 | 880.09 | 14.94 |
| 22.7879 | 919.72 | 15.61 |
| 23.3431 | 121.84 | 2.07 |
| 23.8329 | 618.92 | 10.51 |
| 24.1016 | 2992.56 | 50.80 |
| 24.7982 | 685.38 | 11.64 |
| 25.6629 | 1603.44 | 27.22 |
| 26.7730 | 1490.93 | 25.31 |
| 26.9182 | 2487.28 | 42.23 |
| 27.1711 | 1401.38 | 23.79 |
| 27.4342 | 2391.16 | 40.59 |
| 28.4098 | 978.38 | 16.61 |
| 29.4454 | 1002.79 | 17.02 |
| 29.8441 | 273.66 | 4.65 |
| 30.1732 | 416.26 | 7.07 |
| 30.8702 | 327.30 | 5.56 |
| 30.9955 | 482.52 | 8.19 |
| 32.2250 | 162.51 | 2.76 |
| 32.4730 | 216.94 | 3.68 |
| 34.1810 | 645.27 | 10.95 |
| 34.3229 | 648.62 | 11.01 |

### EXAMPLE 6

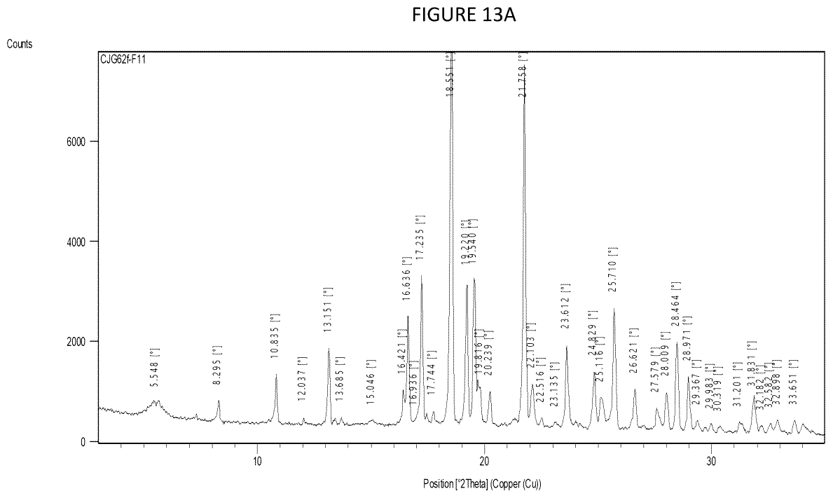

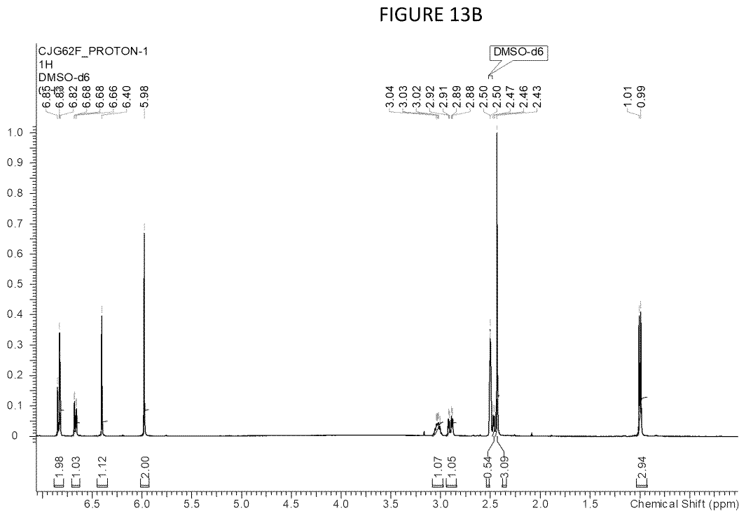

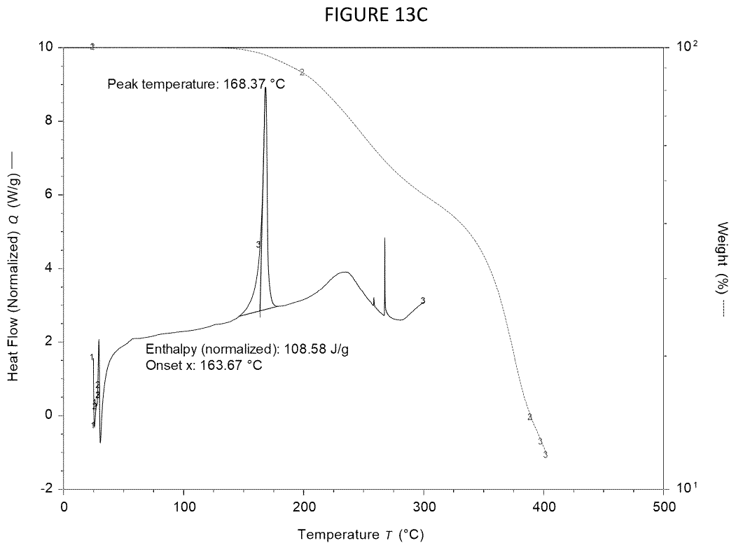

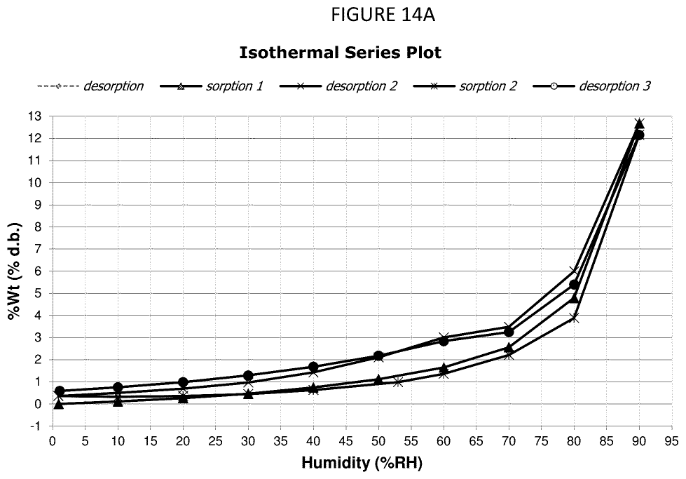

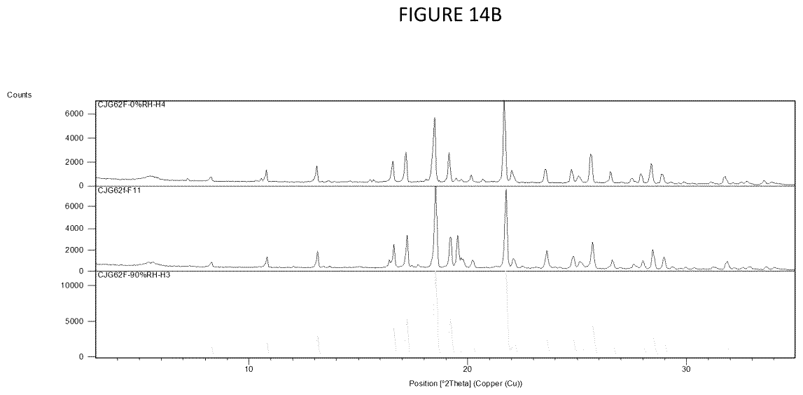

R-MDMA Hemi Fumarate salt pattern A was prepared. An XRPD pattern is shown in FIG. 13A. A ¹H NMR spectrum is shown in FIG. 13B. A combined DSC/TGA thermograph is shown in FIG. 13C. FIG. 14A shows a DVS profile and FIG. 14B shows XRPD patterns at ambient conditions, 0% relative humidity, and 90% relative humidity. TABLE 6 shows a peak list.

TABLE 6

| Pos. [°2Th.] | Height [cts] | Rel. Int. [%] |
|---|---|---|
| 5.5483* | 269.35 | 3.52 |
| 8.2947 | 418.13 | 5.46 |
| 10.8354 | 990.17 | 12.94 |
| 12.0372 | 135.26 | 1.77 |
| 13.1509 | 1529.52 | 19.98 |
| 13.6854 | 155.72 | 2.03 |
| 15.0461 | 104.18 | 1.36 |
| 16.4206 | 735.65 | 9.61 |
| 16.6360 | 2191.31 | 28.63 |
| 16.9358 | 118.80 | 1.55 |
| 17.2348 | 3012.02 | 39.35 |
| 17.7440 | 319.13 | 4.17 |
| 18.5506 | 7654.08 | 100.00 |
| 19.2196 | 2936.93 | 38.37 |
| 19.5403 | 2961.63 | 38.69 |
| 19.8161 | 766.34 | 10.01 |
| 20.2385 | 740.34 | 9.67 |
| 21.7576 | 7237.01 | 94.55 |
| 22.1034 | 901.37 | 11.78 |
| 22.5160 | 223.02 | 2.91 |
| 23.1352 | 136.99 | 1.79 |
| 23.6121 | 1698.78 | 22.19 |
| 24.8289 | 1172.82 | 15.32 |
| 25.1156 | 657.29 | 8.59 |
| 25.7098 | 2446.33 | 31.96 |
| 26.6211 | 842.48 | 11.01 |
| 27.5786 | 463.47 | 6.06 |
| 28.0091 | 792.27 | 10.35 |
| 28.4639 | 1794.27 | 23.44 |
| 28.9710 | 1111.17 | 14.52 |
| 29.3666 | 242.11 | 3.16 |
| 29.9828 | 172.98 | 2.26 |
| 30.3189 | 107.74 | 1.41 |
| 31.2008 | 216.96 | 2.83 |
| 31.8310 | 626.80 | 8.19 |
| 32.1818 | 155.46 | 2.03 |
| 32.5820 | 209.01 | 2.73 |
| 32.8981 | 277.16 | 3.62 |
| 33.6505 | 280.07 | 3.66 |
| 34.0118 | 217.23 | 2.84 |

*The peak at 5.5483°2Theta is due the Kapton film used in the analysis and not related to R-MDMA Hemi Fumarate salt pattern A

### EXAMPLE 7

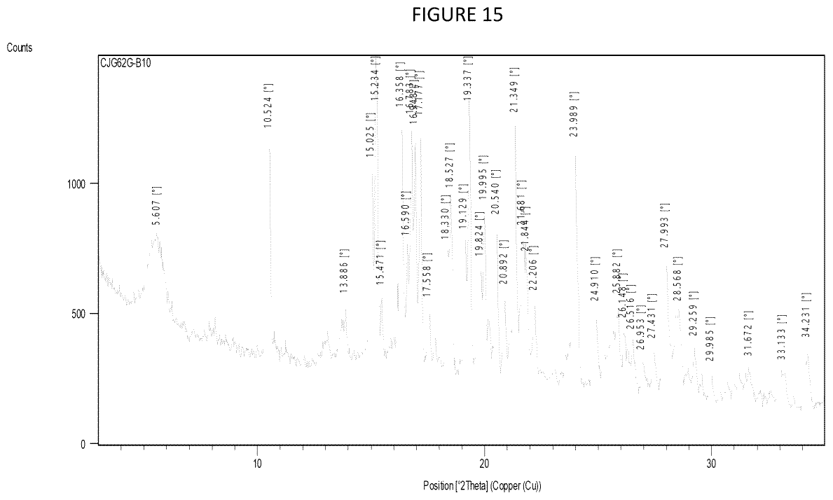

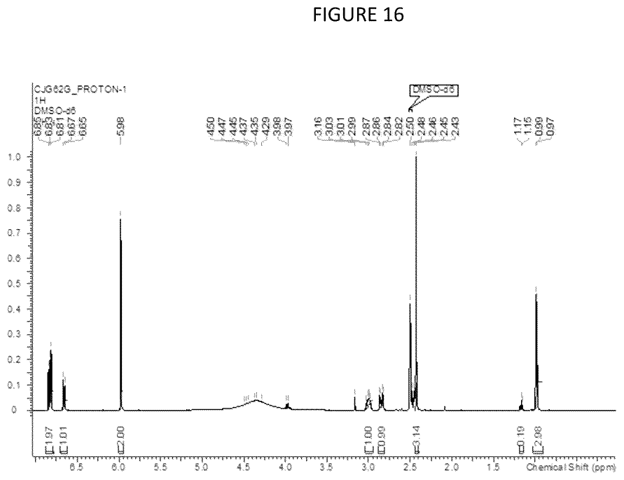

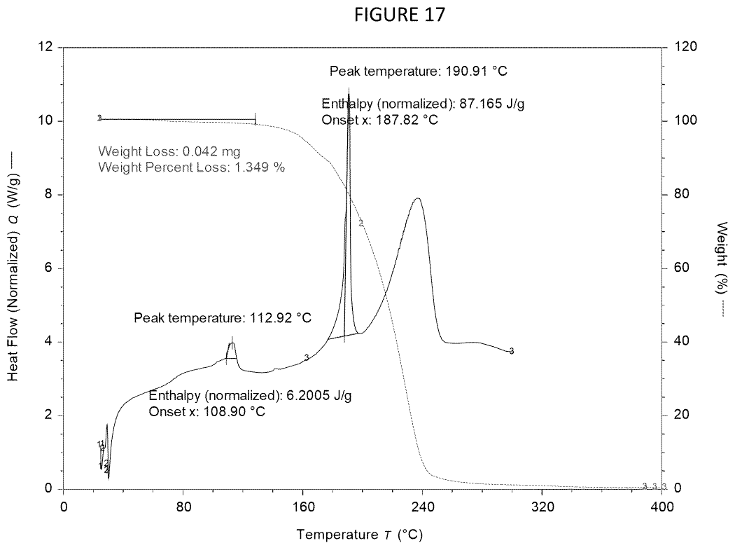

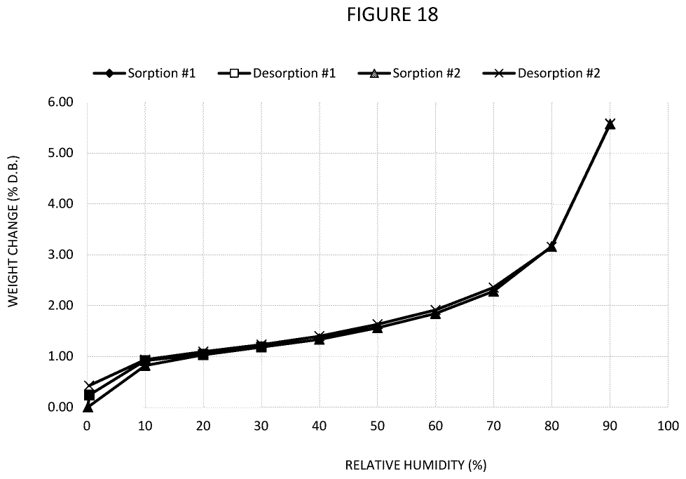

R-MDMA Hemi Oxalate salt pattern A/A′ was prepared. An XRPD pattern is shown in FIG. 15. A ¹H NMR spectrum is shown in FIG. 16. A combined DSC/TGA thermograph is shown in FIG. 17. FIG. 18 shows a DVS profile and FIG. 19 shows XRPD patterns at ambient conditions, 0% relative humidity, and 90% relative humidity. TABLE 7 shows a peak list.

TABLE 7

| Pos. [°2Th.] | Height [cts] | Rel. Int. [%] |
|---|---|---|
| 5.6067* | 286.38 | 23.80 |
| 10.5240 | 814.71 | 67.72 |
| 13.8857 | 217.12 | 18.05 |
| 15.0254 | 750.40 | 62.37 |
| 15.2341 | 1203.14 | 100.00 |
| 15.4706 | 262.50 | 21.82 |
| 16.3578 | 952.81 | 79.19 |
| 16.5902 | 461.18 | 38.33 |
| 16.7826 | 930.69 | 77.36 |
| 16.9482 | 889.44 | 73.93 |
| 17.1771 | 927.09 | 77.06 |
| 17.5576 | 230.64 | 19.17 |
| 18.3303 | 457.58 | 38.03 |
| 18.5269 | 659.50 | 54.81 |
| 19.1292 | 505.95 | 42.05 |
| 19.3374 | 1097.72 | 91.24 |
| 19.8244 | 405.47 | 33.70 |
| 19.9946 | 624.36 | 51.89 |
| 20.5402 | 572.37 | 47.57 |
| 20.8923 | 305.06 | 25.36 |
| 21.3494 | 969.08 | 80.55 |
| 21.6811 | 538.58 | 44.76 |
| 21.8436 | 440.02 | 36.57 |
| 22.2062 | 290.71 | 24.16 |
| 23.9886 | 896.22 | 74.49 |
| 24.9100 | 272.08 | 22.61 |
| 25.8815 | 306.79 | 25.50 |
| 26.1479 | 215.23 | 17.89 |
| 26.5164 | 174.95 | 14.54 |
| 26.9530 | 98.31 | 8.17 |
| 27.4310 | 146.16 | 12.15 |
| 27.9933 | 498.22 | 41.41 |
| 28.5682 | 298.73 | 24.83 |
| 29.2594 | 170.43 | 14.17 |
| 29.9849 | 78.87 | 6.56 |
| 31.6716 | 110.60 | 9.19 |
| 33.1333 | 108.36 | 9.01 |
| 34.2308 | 195.88 | 16.28 |

*The peak at 5.6067°2Theta is due the Kapton film used in the analysis and not related to R-MDMA Hemi Oxalate salt pattern A/A′

### EXAMPLE 8

### Single Crystal X-Ray Structure

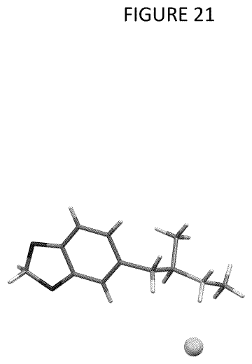

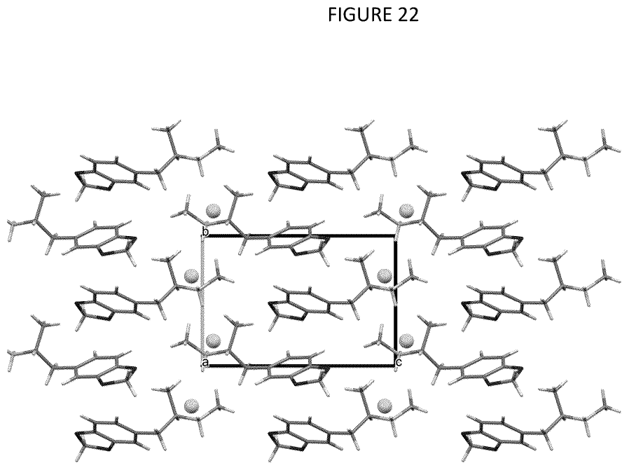

R-MDMA HCl (25 mg) was weighed into a crystallization tube. Dichloromethane (20 vol) was added and the mixture heated to 40° C. The resulting solution was clarified via a 0.45 μm filter and allowed to age allowing for solvent egress. Once suitable crystal growth had occurred, the crystal structure of R-MDMA HCl Form 1 was determined from data measured at low temperature (100 K) and at a wavelength of 1.54180 Å. R-MDMA HCl crystallizes in the monoclinic space group P2₁. In the asymmetric unit, one monocationic (R)-MDMA and one chloride anion were found (overall ratio 1:1) as shown in FIG. 21 and crystal packing was found as shown in FIG. 22.

### EXAMPLE 9

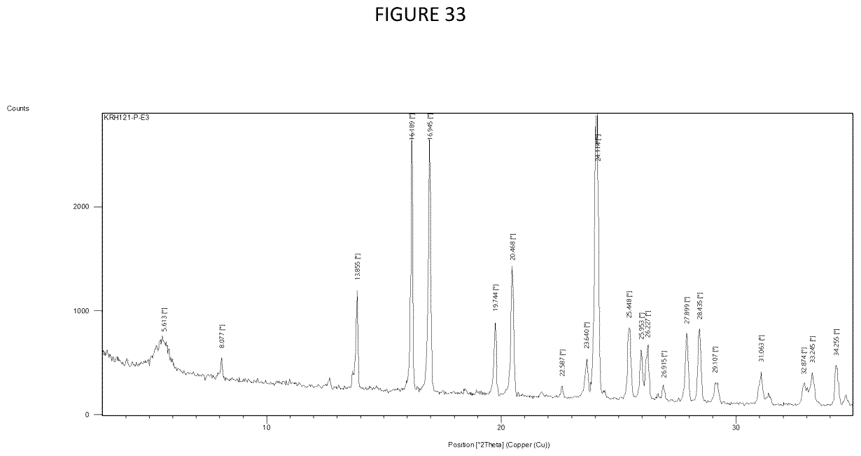

R-MDMA HBr salt pattern B was prepared. TABLE 8 shows XPRD peak data for HBr pattern B. FIG. 33 shows the XPRD pattern.

TABLE 8

| Pos. [°2Theta] | Height [counts] | Rel. Int. [%] |
|---|---|---|
| 5.6128* | 277.76 | 11.09 |
| 8.0771 | 198.08 | 7.91 |
| 13.8548 | 948.75 | 37.87 |
| 16.1891 | 2505.58 | 100.00 |
| 16.9445 | 2433.61 | 97.13 |
| 19.7438 | 710.56 | 28.36 |
| 20.4682 | 1208.93 | 48.25 |
| 22.5868 | 112.60 | 4.49 |
| 23.6399 | 387.01 | 15.45 |
| 24.1138 | 2191.78 | 87.48 |
| 25.4482 | 692.94 | 27.66 |
| 25.9531 | 493.78 | 19.71 |
| 26.2274 | 515.76 | 20.58 |
| 26.9150 | 131.4 | 5.24 |
| 27.8992 | 662.87 | 26.46 |
| 28.4350 | 707.32 | 28.23 |
| 29.1067 | 195.83 | 7.82 |
| 31.0628 | 312.37 | 12.47 |
| 32.8741 | 199.15 | 7.95 |
| 33.2445 | 310.89 | 12.41 |
| 34.2551 | 383.92 | 15.32 |

*The peak at 5.6128°2Theta is due the Kapton film used in the analysis and not related to R-MDMA Hydrobromide salt Pattern B

### EXAMPLE 10

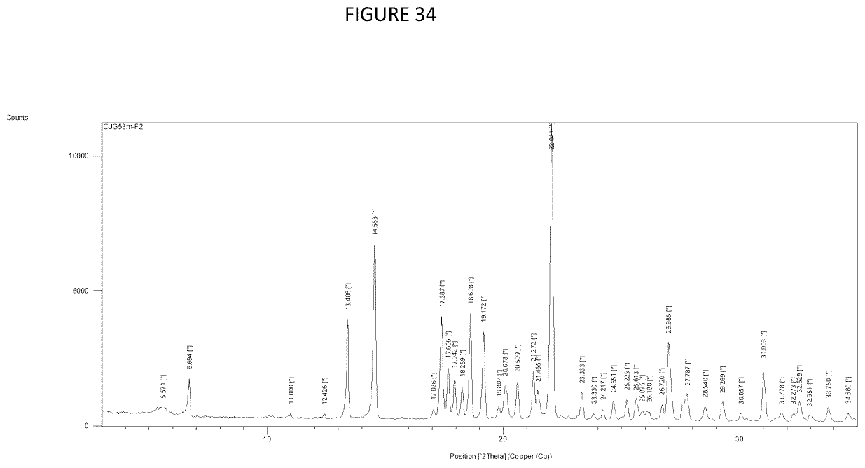

R-MDMA phosphate salt pattern A was prepared. TABLE 9 shows XPRD peak data for phosphate pattern A. FIG. 34 shows the XPRD data.

TABLE 9

| Pos. [°2Th.] | Height [cts] | Rel. Int. [%] |
|---|---|---|
| 5.5661* | 268.91 | 24.32 |
| 13.3505 | 97.94 | 8.86 |
| 14.0295 | 95.08 | 8.60 |
| 14.5108 | 350.8 | 31.73 |
| 15.7641 | 102.62 | 9.28 |
| 17.4001 | 1105.56 | 100.00 |
| 17.9146 | 295.74 | 26.75 |
| 18.2251 | 132.89 | 12.02 |
| 18.5732 | 264.62 | 23.94 |
| 19.1536 | 106.52 | 9.64 |
| 20.6353 | 162.37 | 14.69 |
| 21.9954 | 611.68 | 55.33 |
| 23.2998 | 192.73 | 17.43 |
| 24.6975 | 1066.32 | 96.45 |
| 24.8545 | 637.45 | 57.66 |
| 26.0326 | 100.04 | 9.05 |
| 26.8020 | 294.85 | 26.67 |
| 28.5395 | 68.87 | 6.23 |
| 29.0512 | 144.92 | 13.11 |
| 34.3710 | 68.32 | 6.18 |

*The peak at 5.5661°2Theta is due the Kapton film used in the analysis and not related to R-MDMA Phosphate salt Pattern A

### EXAMPLE 11

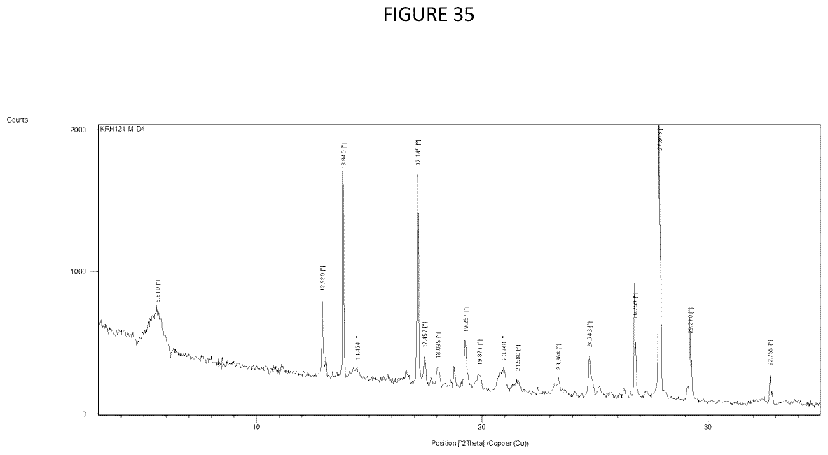

R-MDMA phosphate salt pattern B was prepared. TABLE 10 shows XPRD peak data for phosphate pattern B. FIG. 35 shows the XPRD data.

TABLE 10

| Pos. [°2Th.] | Height [cts] | Rel. Int. [%] |
|---|---|---|
| 5.6096* | 241.62 | 12.9 |
| 12.9201 | 530.82 | 28.34 |
| 13.8398 | 1408.66 | 75.2 |
| 14.4737 | 67.56 | 3.61 |
| 17.1453 | 1474.05 | 78.69 |
| 17.4568 | 192.62 | 10.28 |
| 18.0352 | 126.78 | 6.77 |
| 19.2568 | 325.93 | 17.4 |
| 19.8713 | 100.1 | 5.34 |
| 20.9476 | 148.48 | 7.93 |
| 21.5796 | 78.41 | 4.19 |
| 23.3679 | 104.17 | 5.56 |
| 24.7430 | 273.21 | 14.58 |
| 26.7586 | 490.38 | 26.18 |
| 27.8429 | 1873.25 | 100 |
| 29.2104 | 407.13 | 21.73 |
| 32.7550 | 200.23 | 10.69 |

*The peak at 5.6096°2Theta is due the Kapton film used in the analysis and not related to R-MDMA Phosphate salt Pattern B

### EXAMPLE 12

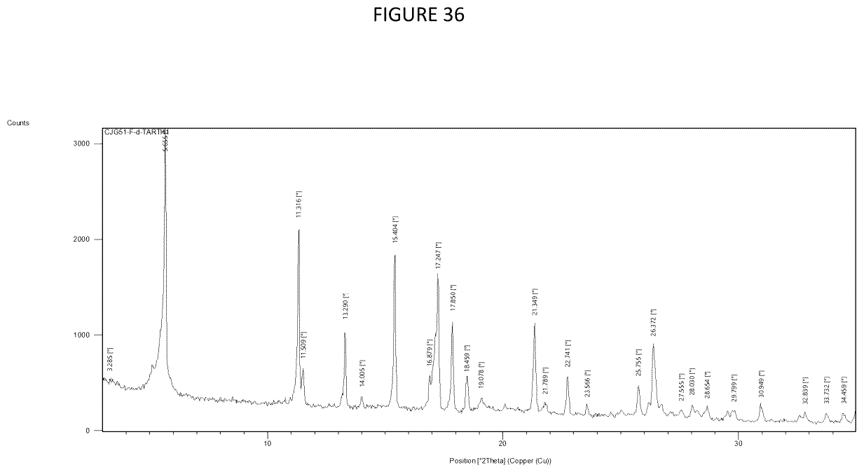

R-MDMA tartrate salt pattern A was prepared. TABLE 11 shows XPRD peak data for tartrate pattern A. FIG. 36 shows the XPRD data.

TABLE 11

| Pos. [°2Th.] | Height [cts] | Rel. Int. [%] |
|---|---|---|
| 3.2852 | 180.25 | 6.37 |
| 5.6552 | 2830.79 | 100.00 |
| 11.3164 | 1860.27 | 65.72 |
| 11.5095 | 384.03 | 13.57 |
| 13.2902 | 817.14 | 28.87 |
| 14.0055 | 117.25 | 4.14 |
| 15.4044 | 1631.13 | 57.62 |
| 16.8794 | 352.65 | 12.46 |
| 17.2467 | 1375.03 | 48.57 |
| 17.8497 | 938.46 | 33.15 |
| 18.4594 | 310.23 | 10.96 |
| 19.0778 | 138.72 | 4.90 |
| 21.3487 | 925.62 | 32.70 |
| 21.7886 | 105.41 | 3.72 |
| 22.7414 | 396.37 | 14.00 |
| 23.5663 | 91.55 | 3.23 |
| 25.7552 | 327.96 | 11.59 |
| 26.3722 | 751.63 | 26.55 |
| 27.5549 | 83.66 | 2.96 |
| 28.0300 | 147.14 | 5.20 |
| 28.6545 | 118.65 | 4.19 |
| 29.7989 | 95.18 | 3.36 |
| 30.9490 | 142.05 | 5.02 |
| 32.8387 | 79.81 | 2.82 |
| 33.7321 | 88.26 | 3.12 |
| 34.4586 | 84.37 | 2.98 |

### EXAMPLE 13

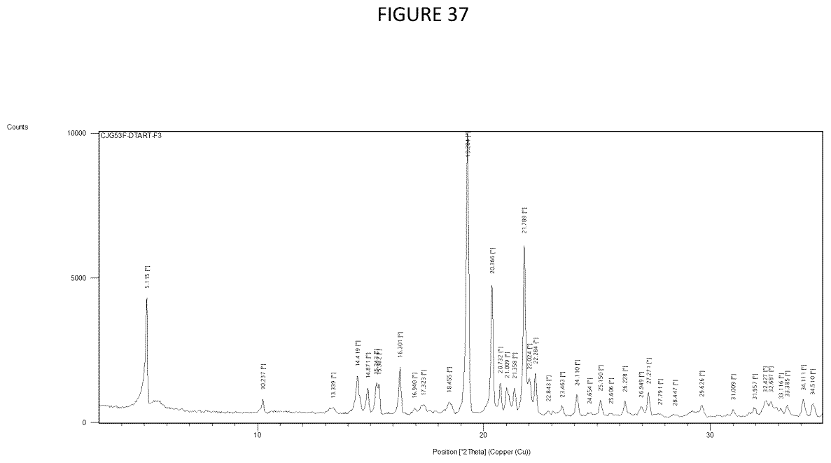

R-MDMA tartrate salt pattern B was prepared. TABLE 12 shows XPRD peak data for tartrate pattern B. FIG. 37 shows the XPRD data.

TABLE 12

| Pos. [°2Th.] | Height [cts] | Rel. Int. [%] |
|---|---|---|
| 5.1154 | 3836.59 | 39.19 |
| 10.2367 | 452.95 | 4.63 |
| 13.3392 | 165.32 | 1.69 |
| 14.4193 | 1284.46 | 13.12 |
| 14.8706 | 875.43 | 8.94 |
| 15.2430 | 1009.91 | 10.32 |
| 15.3823 | 997.71 | 10.19 |
| 16.3008 | 1600.39 | 16.35 |
| 16.9403 | 200.64 | 2.05 |
| 17.3228 | 305.39 | 3.12 |
| 18.4550 | 411.49 | 4.20 |
| 19.2838 | 9790.36 | 100.00 |
| 20.3663 | 4552.07 | 46.5 |
| 20.7318 | 1112.48 | 11.36 |
| 21.0086 | 956.01 | 9.76 |
| 21.3581 | 927.69 | 9.48 |
| 21.7890 | 5956.64 | 60.84 |
| 22.0242 | 1250.14 | 12.77 |
| 22.2842 | 1441.90 | 14.73 |
| 22.8432 | 143.22 | 1.46 |
| 23.4631 | 290.74 | 2.97 |
| 24.1102 | 705.71 | 7.21 |
| 24.6539 | 71.37 | 0.73 |
| 25.1504 | 525.50 | 5.37 |
| 25.6056 | 80.56 | 0.82 |
| 26.2284 | 516.33 | 5.27 |
| 26.9493 | 317.97 | 3.25 |
| 27.2705 | 795.07 | 8.12 |
| 27.7912 | 73.29 | 0.75 |
| 28.4469 | 39.49 | 0.40 |
| 29.6258 | 363.6 | 3.71 |
| 31.0092 | 233.51 | 2.39 |
| 31.9570 | 251.63 | 2.57 |
| 32.4271 | 510.96 | 5.22 |
| 32.6872 | 508.76 | 5.20 |
| 33.1162 | 267.26 | 2.73 |
| 33.3847 | 360.55 | 3.68 |
| 34.1111 | 573.99 | 5.86 |
| 34.5103 | 412.42 | 4.21 |

### EXAMPLE 14

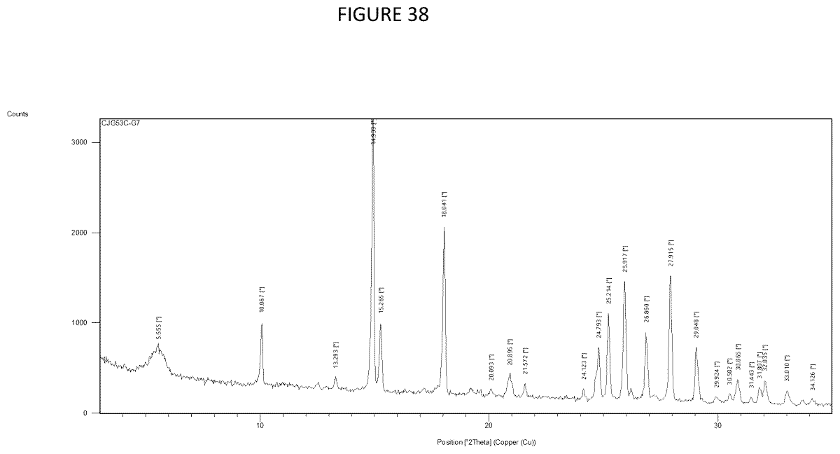

R-MDMA maleate salt pattern A was prepared. TABLE 13 shows XPRD peak data for maleate pattern A. FIG. 38 shows the XPRD data.

TABLE 13

| Pos. [°2Th.] | Height [cts] | Rel. Int. [%] |
|---|---|---|
| 5.5552* | 273.38 | 8.75 |
| 10.0668 | 692.83 | 22.17 |
| 13.2926 | 129.00 | 4.13 |
| 14.9329 | 3124.99 | 100.00 |
| 15.2652 | 759.40 | 24.30 |
| 18.0407 | 1852.08 | 59.27 |
| 20.0928 | 64.96 | 2.08 |
| 20.8947 | 241.03 | 7.71 |
| 21.5717 | 150.79 | 4.83 |
| 24.1232 | 114.63 | 3.67 |
| 24.7935 | 578.76 | 18.52 |
| 25.2140 | 956.81 | 30.62 |
| 25.9166 | 1320.52 | 42.26 |
| 26.8599 | 760.13 | 24.32 |
| 27.9146 | 1394.9 | 44.64 |
| 29.0481 | 606.03 | 19.39 |
| 29.9241 | 56.38 | 1.80 |
| 30.5025 | 95.43 | 3.05 |
| 30.8645 | 260.87 | 8.35 |
| 31.4425 | 61.83 | 1.98 |
| 31.8066 | 172.01 | 5.5 |
| 32.0353 | 258.38 | 8.27 |
| 33.0096 | 146.53 | 4.69 |
| 34.1256 | 54.76 | 1.75 |

*The peak at 5.5552°2Theta is due the Kapton film used in the analysis and not related to R-MDMA Maleate salt Pattern A

### EXAMPLE 15

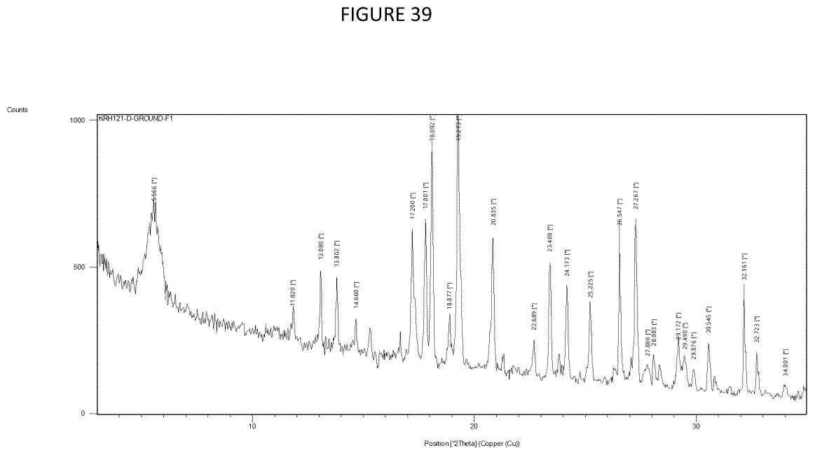

R-MDMA L-malate salt pattern A was prepared. TABLE 14 shows XPRD peak data for L-maleate pattern A. FIG. 39 shows the XPRD data.

TABLE 14

| Pos. [°2Th.] | Height [cts] | Rel. Int. [%] |
|---|---|---|
| 5.5662* | 292.17 | 32.94 |
| 11.8203 | 89.59 | 10.10 |
| 13.0800 | 267.20 | 30.13 |
| 13.8024 | 250.56 | 28.25 |
| 14.6599 | 118.72 | 13.39 |
| 17.1997 | 448.66 | 50.59 |
| 17.8007 | 488.07 | 55.03 |
| 18.0922 | 753.52 | 84.97 |
| 18.8774 | 156.25 | 17.62 |
| 19.2735 | 886.85 | 100.00 |
| 20.8354 | 459.27 | 51.79 |
| 22.6889 | 117.28 | 13.22 |
| 23.4077 | 392.35 | 44.24 |
| 24.1732 | 315.49 | 35.57 |
| 25.2252 | 248.48 | 28.02 |
| 26.5473 | 505.03 | 56.95 |
| 27.2672 | 563.85 | 63.58 |
| 27.8003 | 66.12 | 7.46 |
| 28.0832 | 103.59 | 11.68 |
| 29.1725 | 132.38 | 14.93 |
| 29.4901 | 100.28 | 11.31 |
| 29.8761 | 69.89 | 7.88 |
| 30.5450 | 162.99 | 18.38 |
| 32.1611 | 355.32 | 40.07 |
| 32.7232 | 145.17 | 16.37 |
| 34.0007 | 37.84 | 4.27 |

*The peak at 5.5662°2Theta is due the Kapton film used in the analysis and not related to R-MDMA L-Malate salt Pattern A

### EXAMPLE 16

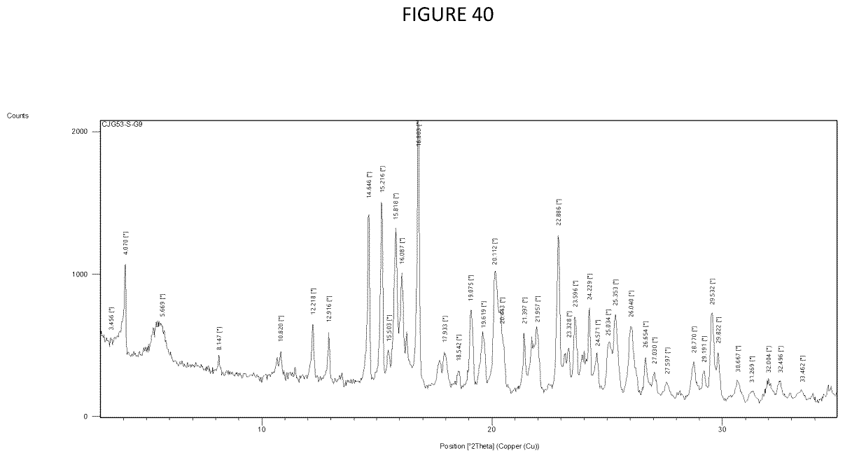

R-MDMA hemi-napthylene-1,5-disulfonate salt pattern A was prepared. TABLE 15 shows XPRD peak data for hemi-napthylene-1,5-disulfonate pattern A. FIG. 40 shows the XPRD data.

TABLE 15

| Pos. [°2Th.] | Height [cts] | Rel. Int. [%] |
|---|---|---|
| 3.4561 | 419.96 | 21.00 |
| 4.0704 | 949.76 | 47.5 |
| 5.6688* | 513.72 | 25.69 |
| 8.1471 | 280.45 | 14.03 |
| 10.8199 | 335.14 | 16.76 |
| 12.2179 | 527.28 | 26.37 |
| 12.9164 | 470.99 | 23.56 |
| 14.6460 | 1342.90 | 67.17 |
| 15.2155 | 1383.32 | 69.19 |
| 15.5027 | 337.18 | 16.86 |
| 15.8180 | 1195.41 | 59.79 |
| 16.0875 | 882.96 | 44.16 |
| 16.8031 | 1999.40 | 100.00 |
| 17.9332 | 330.84 | 16.55 |
| 18.5420 | 202.07 | 10.11 |
| 19.0754 | 620.78 | 31.05 |
| 19.6194 | 436.41 | 21.83 |
| 20.1116 | 881.05 | 44.07 |
| 20.4433 | 463.25 | 23.17 |
| 21.3968 | 463.79 | 23.2 |
| 21.9571 | 471.27 | 23.57 |
| 22.8863 | 1155.37 | 57.79 |
| 23.3282 | 365.48 | 18.28 |
| 23.5957 | 580.06 | 29.01 |
| 24.2285 | 630.66 | 31.54 |
| 24.5715 | 307.64 | 15.39 |
| 25.0337 | 380.22 | 19.02 |
| 25.3534 | 590.37 | 29.53 |
| 26.0402 | 510.36 | 25.53 |
| 26.6543 | 294.38 | 14.72 |
| 27.0299 | 183.06 | 9.16 |
| 27.5965 | 112.22 | 5.61 |
| 28.7697 | 261.99 | 13.10 |
| 29.1905 | 191.75 | 9.59 |
| 29.5317 | 597.13 | 29.87 |
| 29.8224 | 326.38 | 16.32 |
| 30.6671 | 128.02 | 6.40 |
| 31.2694 | 53.00 | 2.65 |
| 32.0042 | 120.94 | 6.05 |
| 32.4960 | 121.27 | 6.07 |
| 33.4625 | 55.65 | 2.78 |

*The peak at 5.6688°2Theta is due the Kapton film used in the analysis and not related to R-MDMA Hemi-napthylene-1,5-disulfonate salt Pattern A

### EXAMPLE 17

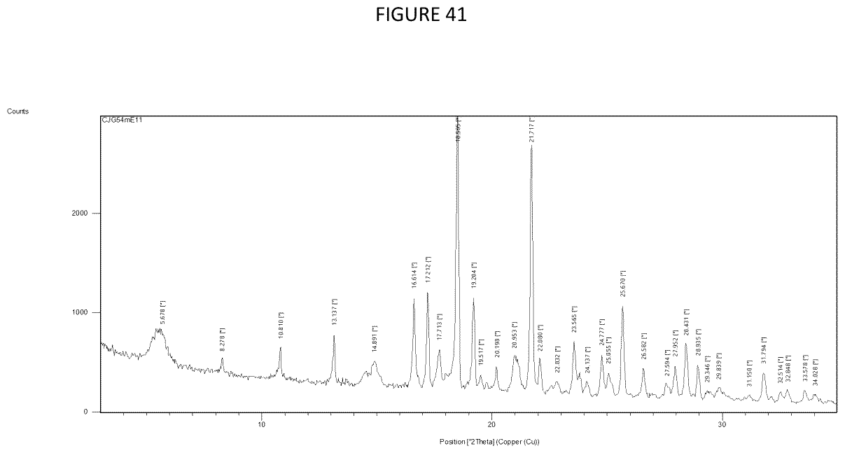

R-MDMA hemi-fumarate salt pattern A was prepared. TABLE 16 shows XPRD peak data for hemi-fumarate pattern A. FIG. 41 shows the data.

TABLE 16

| Pos. [°2Th.] | Height [cts] | Rel. Int. [%] |
|---|---|---|
| 5.6776* | 292.45 | 10.66 |
| 8.2781 | 130.39 | 4.75 |
| 10.8097 | 318.67 | 11.61 |
| 13.1373 | 489.36 | 17.83 |
| 14.8913 | 236.12 | 8.61 |
| 16.6141 | 892.35 | 32.52 |
| 17.2125 | 958.49 | 34.93 |
| 17.7134 | 382.08 | 13.92 |
| 18.5047 | 2743.94 | 100.00 |
| 19.2044 | 916.14 | 33.39 |
| 19.5173 | 145.41 | 5.30 |
| 20.1977 | 233.44 | 8.51 |
| 20.9529 | 330.48 | 12.04 |
| 21.7168 | 2519.34 | 91.81 |
| 22.0799 | 316.38 | 11.53 |
| 22.8318 | 102.23 | 3.73 |
| 23.5645 | 511.81 | 18.65 |
| 24.1374 | 108.44 | 3.95 |
| 24.7768 | 392.14 | 14.29 |
| 25.0545 | 210.88 | 7.69 |
| 25.6702 | 893.91 | 32.58 |
| 26.5823 | 270.95 | 9.87 |
| 27.5938 | 114.09 | 4.16 |
| 27.9523 | 314.15 | 11.45 |
| 28.4305 | 528.14 | 19.25 |
| 28.9354 | 333.05 | 12.14 |
| 29.346 | 65.45 | 2.39 |
| 29.8385 | 102.76 | 3.75 |
| 31.1497 | 41.65 | 1.52 |
| 31.7937 | 281.98 | 10.28 |
| 32.514 | 85.19 | 3.10 |
| 32.8481 | 106.13 | 3.87 |
| 33.5784 | 119.65 | 4.36 |
| 34.0279 | 79.15 | 2.88 |

*The peak at 5.6776°2Theta is due the Kapton film used in the analysis and not related to R-MDMA Hemi-fumarate salt Pattern A

### EXAMPLE 18

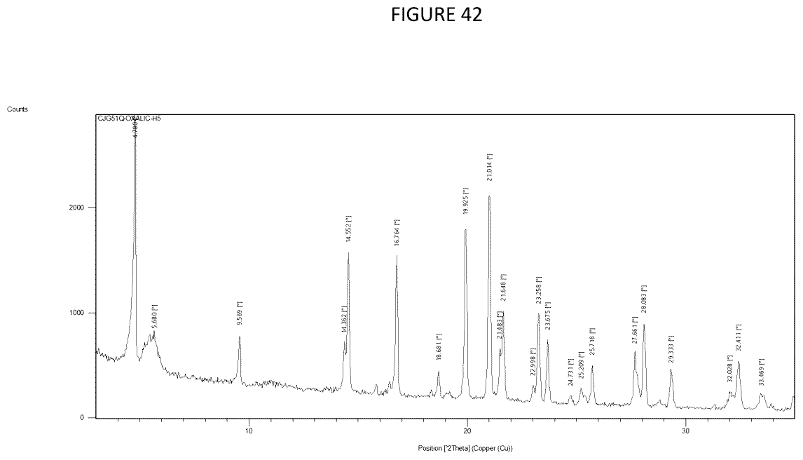

R-MDMA oxalate salt pattern A was prepared. TABLE 17 shows XPRD peak data for oxalate salt pattern A. FIG. 42 shows the data.

TABLE 17

| Pos. [°2Th.] | Height [cts] | Rel. Int. [%] |
|---|---|---|
| 4.7803 | 2417.01 | 100.00 |
| 5.6800* | 301.49 | 12.47 |
| 9.5688 | 466.34 | 19.29 |
| 14.3615 | 461.24 | 19.08 |
| 14.5524 | 1323.96 | 54.78 |
| 16.7644 | 1323.05 | 54.74 |
| 18.6813 | 255.38 | 10.57 |
| 19.9255 | 1654.73 | 68.46 |
| 21.0140 | 1991.20 | 82.38 |
| 21.4829 | 494.49 | 20.46 |
| 21.6477 | 856.19 | 35.42 |
| 22.9982 | 160.71 | 6.65 |
| 23.2578 | 847.49 | 35.06 |
| 23.6747 | 603.85 | 24.98 |
| 24.7315 | 78.83 | 3.26 |
| 25.2086 | 150.68 | 6.23 |
| 25.7177 | 377.44 | 15.62 |
| 27.6607 | 502.72 | 20.80 |
| 28.0826 | 799.42 | 33.07 |
| 29.3331 | 331.69 | 13.72 |
| 32.0277 | 157.76 | 6.53 |
| 32.4106 | 450.15 | 18.62 |
| 33.4686 | 123.06 | 5.09 |

*The peak at 5.6800°2Theta is due the Kapton film used in the analysis and not related to R-MDMA Oxalate salt Pattern A

### EXAMPLE 19

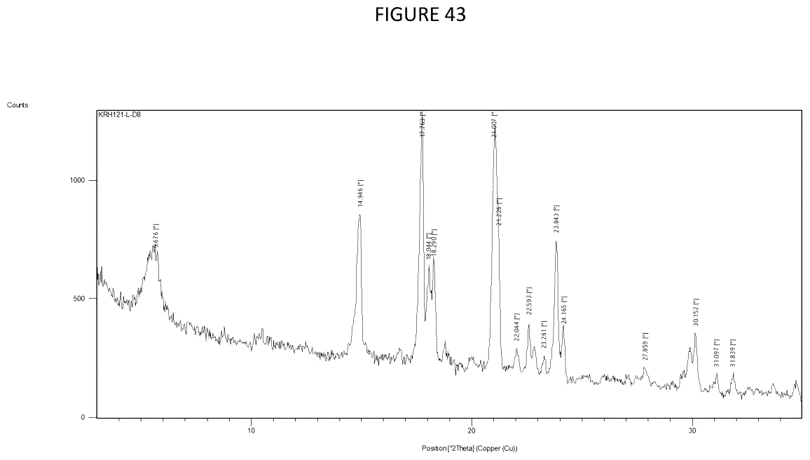

R-MDMA sulfate salt pattern A was prepared. TABLE 18 shows XPRD peak data for sulfate pattern A. FIG. 43 shows the data.

TABLE 18

| Pos. [°2Th.] | Height [cts] | Rel. Int. [%] |
|---|---|---|
| 5.6758* | 245.26 | 23.88 |
| 14.9463 | 600.95 | 58.50 |
| 17.7631 | 1027.23 | 100.00 |
| 18.0436 | 405.51 | 39.48 |
| 18.2903 | 423.73 | 41.25 |
| 21.0072 | 950.85 | 92.56 |
| 21.2257 | 579.52 | 56.42 |
| 22.0438 | 98.43 | 9.58 |
| 22.5929 | 214.51 | 20.88 |
| 23.2614 | 78.60 | 7.65 |
| 23.8432 | 572.53 | 55.74 |
| 24.1647 | 189.39 | 18.44 |
| 27.8586 | 61.45 | 5.98 |
| 30.1519 | 223.72 | 21.78 |
| 31.0970 | 60.78 | 5.92 |
| 31.8386 | 64.94 | 6.32 |

*The peak at 5.6758°2Theta is due the Kapton film used in the analysis and not related to R-MDMA Sulfate salt Pattern A

### EXAMPLE 20

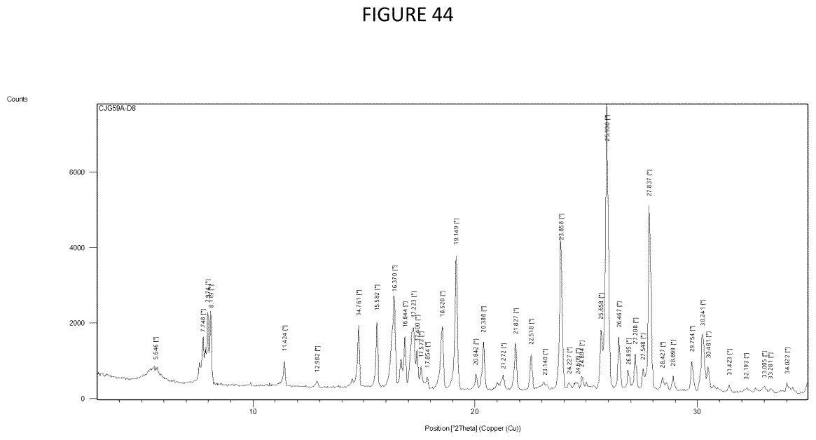

R-MDMA sulfate salt pattern B was prepared. TABLE 19 shows XPRD peak data for sulfate pattern B. FIG. 44 shows the data.

TABLE 19

| Pos. [°2Th.] | Height [cts] | Rel. Int. [%] |
|---|---|---|
| 5.6459* | 312.53 | 4.91 |
| 7.7484 | 1117.07 | 17.56 |
| 7.9744 | 1907.59 | 30.00 |
| 8.1187 | 1775.86 | 27.92 |
| 11.4236 | 670.82 | 10.55 |
| 12.9016 | 147.98 | 2.33 |
| 14.7607 | 1643.5 | 25.84 |
| 15.5820 | 1795.78 | 28.24 |
| 16.3703 | 2304.95 | 36.24 |
| 16.8435 | 1357.73 | 21.35 |
| 17.2234 | 1612.83 | 25.36 |
| 17.3999 | 996.25 | 15.67 |
| 17.5729 | 569.62 | 8.96 |
| 17.8540 | 292.85 | 4.6 |
| 18.5197 | 1650.96 | 25.96 |
| 19.1489 | 3573.65 | 56.19 |
| 20.0420 | 384.33 | 6.04 |
| 20.3879 | 1245.65 | 19.59 |
| 21.2722 | 284.38 | 4.47 |
| 21.8271 | 1209.53 | 19.02 |
| 22.5176 | 934.77 | 14.70 |
| 23.1398 | 132.61 | 2.09 |
| 23.8579 | 3730.92 | 58.67 |
| 24.2268 | 190.38 | 2.99 |
| 24.6091 | 167.57 | 2.63 |
| 24.8039 | 367.95 | 5.79 |
| 25.6585 | 1630.84 | 25.64 |
| 25.9377 | 6359.64 | 100.00 |
| 26.4666 | 1409.84 | 22.17 |
| 26.8953 | 433.68 | 6.82 |
| 27.2082 | 986.72 | 15.52 |
| 27.5481 | 570.12 | 8.96 |
| 27.8372 | 4908.13 | 77.18 |
| 28.4267 | 362.82 | 5.71 |
| 28.8986 | 408.14 | 6.42 |
| 29.7543 | 795.98 | 12.52 |
| 30.2410 | 1484 | 23.33 |
| 30.4810 | 637.72 | 10.03 |
| 31.4228 | 187.52 | 2.95 |
| 32.1925 | 86.40 | 1.36 |
| 33.0050 | 131.52 | 2.07 |
| 33.2811 | 104.29 | 1.64 |
| 34.0216 | 249.87 | 3.93 |

*The peak at 5.6459°2Theta is due the Kapton film used in the analysis and not related to R-MDMA Sulfate salt Pattern B

### EXAMPLE 21

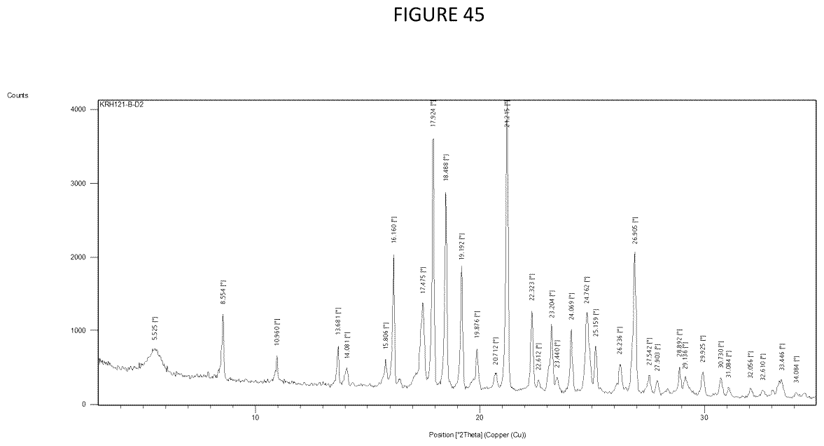

R-MDMA mesylate salt pattern A was prepared. TABLE 20 shows XPRD peak data for mesylate pattern A. FIG. 45 shows the data.

TABLE 20

| Pos. [°2Th.] | Height [cts] | Rel. Int. [%] |
|---|---|---|
| 5.5246* | 308.75 | 7.87 |
| 8.5544 | 890.95 | 22.71 |
| 10.9603 | 373.51 | 9.52 |
| 13.6810 | 547.78 | 13.97 |
| 14.0813 | 227.27 | 5.79 |
| 15.8062 | 370.07 | 9.43 |
| 16.1599 | 1815.73 | 46.29 |
| 17.4746 | 1138.46 | 29.02 |
| 17.9244 | 3500.9 | 89.25 |
| 18.4882 | 2681.18 | 68.36 |
| 19.1923 | 1611.76 | 41.09 |
| 19.8760 | 559.39 | 14.26 |
| 20.7120 | 211.15 | 5.38 |
| 21.2153 | 3922.40 | 100.00 |
| 22.3234 | 1089.78 | 27.78 |
| 22.6119 | 155.68 | 3.97 |
| 23.2038 | 872.94 | 22.26 |
| 23.4404 | 196.27 | 5 |
| 24.0694 | 847.93 | 21.62 |
| 24.7619 | 1077.11 | 27.46 |
| 25.1591 | 635.58 | 16.20 |
| 26.2361 | 404.18 | 10.30 |
| 26.9048 | 1908.51 | 48.66 |
| 27.5418 | 264.59 | 6.75 |
| 27.9032 | 187.77 | 4.79 |
| 28.8917 | 396.43 | 10.11 |
| 29.1362 | 222.28 | 5.67 |
| 29.9252 | 321.78 | 8.20 |
| 30.7305 | 250.76 | 6.39 |
| 31.0839 | 125.67 | 3.20 |
| 32.0562 | 122.81 | 3.13 |
| 32.6099 | 91.73 | 2.34 |
| 33.4459 | 225.43 | 5.75 |
| 34.0844 | 60.22 | 1.54 |

*The peak at 5.5246°2Theta is due the Kapton film used in the analysis and not related to R-MDMA Mesylate salt Pattern A

### EXAMPLE 22

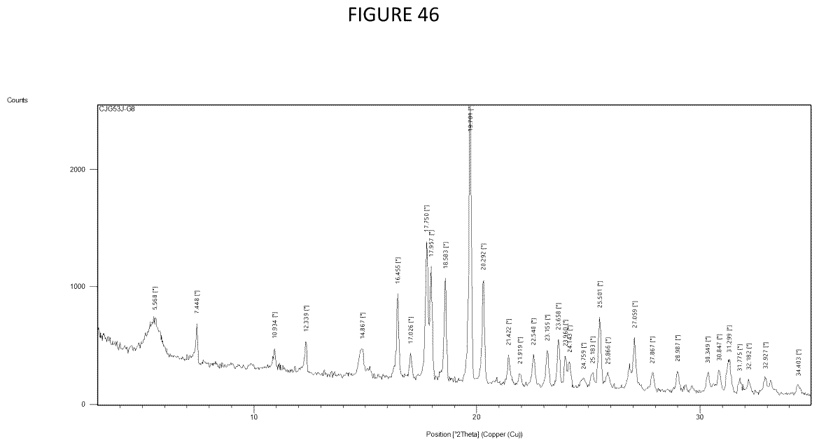

R-MDMA acetate salt pattern A was prepared. TABLE 21 shows XPRD peak data for acetate pattern A. FIG. 46 shows the data.

TABLE 21

| Pos. [°2Th.] | Height [cts] | Rel. Int. [%] |
|---|---|---|
| 5.5678* | 287.37 | 12.19 |
| 7.4482 | 325.60 | 13.81 |
| 10.9336 | 183.68 | 7.79 |
| 12.3386 | 265.46 | 11.26 |
| 14.8665 | 237.00 | 10.05 |
| 16.4549 | 720.48 | 30.57 |
| 17.0256 | 216.59 | 9.19 |
| 17.7496 | 1175.31 | 49.86 |
| 17.9574 | 967.20 | 41.03 |
| 18.5827 | 868.02 | 36.83 |
| 19.7005 | 2357.05 | 100.00 |
| 20.2921 | 873.47 | 37.06 |
| 21.4221 | 238.37 | 10.11 |
| 21.9194 | 88.32 | 3.75 |
| 22.5404 | 254.49 | 10.8 |
| 23.155 | 294.93 | 12.51 |
| 23.6578 | 393.98 | 16.71 |
| 23.9598 | 253.17 | 10.74 |
| 24.1429 | 208.33 | 8.84 |
| 24.7590 | 66.15 | 2.81 |
| 25.1835 | 119.82 | 5.08 |
| 25.5008 | 598.96 | 25.41 |
| 25.8655 | 116.53 | 4.94 |
| 27.0594 | 440.07 | 18.67 |
| 27.8674 | 145.03 | 6.15 |
| 28.9870 | 165.14 | 7.01 |
| 30.3487 | 165.25 | 7.01 |
| 30.8472 | 185.74 | 7.88 |
| 31.2993 | 260.10 | 11.04 |
| 31.7746 | 105.32 | 4.47 |
| 32.1819 | 116.80 | 4.96 |
| 32.9273 | 130.61 | 5.54 |
| 34.4031 | 78.06 | 3.31 |

*The peak at 5.5678°2Theta is due the Kapton film used in the analysis and not related to R-MDMA Acetate salt Pattern A

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

What is claimed is:

1. A composition of a crystalline form salt or polymorph of R-MDMA.

2. The composition of claim 1, wherein said salt is chosen from the group consisting of hydrochloride, hydrobromide, maleate, L-malate, D-tartrate, hemi-meso-tartrate, hemi-L-tartrate, citrate, phosphate, hemi-naphthylene-1,5-disulphonate, hemi-fumarate, sulfate, mesylate, acetate, hemi-oxalate, and oxalate.

3. The composition of claim 1, wherein said salt is chosen from the group consisting of hydrochloride pattern A, phosphate pattern A, phosphate pattern B, phosphate pattern C, HBr pattern A, HBr pattern B, HBr pattern C, hemi-L-tartrate pattern A, hemi-meso-tartrate pattern B, hemi-meso-tartrate pattern C, meso-tartrate pattern A, meso-tartrate pattern B, sulfate pattern A, sulfate pattern B, D-tartrate pattern A, D-tartrate pattern B, D-tartrate pattern C, D-tartrate pattern D, D-tartrate pattern E, L-maleate pattern A, maleate pattern A, maleate pattern B, hemi naptheylene-1,5-disulfonate pattern A, hemi naptheylene-1,5-disulfonate pattern B, hemi-oxalate pattern A, hemi-oxalate pattern A′, hemi-fumarate pattern A, hemi-fumarate pattern A′, mesylate pattern A, acetate pattern A, citrate pattern A, fumarate pattern A, and oxalate pattern A.

4. The composition of claim 1, wherein said composition is in the form of a prodrug.

5. The composition of claim 4, wherein said prodrug is an amino acid covalently attached to said crystalline form salt or polymorph of R-MDMA.

6. The composition of claim 5, wherein said amino acid is chosen from the group consisting of lysine, alanine, arginine, asparagine, aspartic acid, cysteine, glutamine, glutamic acid, glycine, histidine, isoleucine, leucine, methionine, phenylalanine, proline, serine, threonine, tryptophan, tyrosine, and valine.

7. A pharmaceutical composition comprising a crystalline form salt or polymorph of R-MDMA and pharmaceutically acceptable excipients.

8. The pharmaceutical composition of claim 7, wherein said salt is chosen from the group consisting of hydrochloride, hydrobromide, maleate, L-malate, D-tartrate, hemi-meso-tartrate, hemi-L-tartrate, citrate, phosphate, hemi-naphthylene-1,5-disulphonate, hemi-fumarate, sulfate, mesylate, acetate, hemi-oxalate, and oxalate.

9. The pharmaceutical composition of claim 7, wherein said salt is chosen from the group consisting of hydrochloride pattern A, phosphate pattern A, phosphate pattern B, phosphate pattern C, HBr pattern A, HBr pattern B, HBr pattern C, hemi-L-tartrate pattern A, hemi-meso-tartrate pattern B, hemi-meso-tartrate pattern C, meso-tartrate pattern A, meso-tartrate pattern B, sulfate pattern A, sulfate pattern B, D-tartrate pattern A, D-tartrate pattern B, D-tartrate pattern C, D-tartrate pattern D, D-tartrate pattern E, L-maleate pattern A, maleate pattern A, maleate pattern B, hemi naptheylene-1,5-disulfonate pattern A, hemi naptheylene-1,5-disulfonate pattern B, hemi-oxalate pattern A, hemi-oxalate pattern A′, hemi-fumarate pattern A, hemi-fumarate pattern A′, mesylate pattern A, acetate pattern A, citrate pattern A, fumarate pattern A, and oxalate pattern A.

10. The pharmaceutical composition of claim 7, wherein said composition is in the form of a prodrug.

11. The composition of claim 10, wherein said prodrug is an amino acid covalently attached to said crystalline form salt or polymorph of R-MDMA.

12. The pharmaceutical composition of claim 11, wherein said amino acid is chosen from the group consisting of lysine, alanine, arginine, asparagine, aspartic acid, cysteine, glutamine, glutamic acid, glycine, histidine, isoleucine, leucine, methionine, phenylalanine, proline, serine, threonine, tryptophan, tyrosine, and valine.

13. The pharmaceutical composition of claim 7, wherein said composition is formulated in a continual slow-release formulation.

14. The pharmaceutical composition of claim 13, wherein said composition is formulated in a transdermal patch.

15. The pharmaceutical composition of claim 7, wherein said composition is formulated in an intranasal spray.

16. The pharmaceutical composition of claim 7, wherein said composition is formulated in a liquid dosage form chosen from the group consisting of suspensions, solutions, emulsions, elixirs, tinctures, sprays, syrups, gels, magmas, liniments, lotions, ointments, pastes, drops, and inhalants.

17. The pharmaceutical composition of claim 7, wherein said composition is formulated in a solid dosage form chosen from the group consisting of capsules, films, lozenge, patch, powder, tablets, pellets, pills, and troches.

18. A method of treating an individual for a medical condition, including the steps of:
administering an effective amount of a composition of a crystalline form salt or polymorph of R-MDMA to the individual; and
treating the individual.

19. The method of claim 18, further including the step of preventing or reducing side effects of neurotoxicity, hyperthermia and dependence/addiction experienced with racemic MDMA.

20. The method of claim 18, wherein the medical condition is chosen from the group consisting of post-traumatic stress disorder, social anxiety, autism spectrum disorder, substance use disorder, depression, anxiety disorder, anxiety with life-threatening disease, personality disorder, schizophrenia, obsessive compulsive disorder, couple therapy, enhancement of any psychotherapy by inducing feelings of well-being connectivity, trust, love, empathy, openness, and pro-sociality, and enhancing therapeutic bond in any psychotherapy of patients or neurotic/healthy subjects.

21. The method of claim 18, wherein the salt is chosen from the group consisting of hydrochloride, hydrobromide, maleate, L-malate, D-tartrate, hemi-meso-tartrate, hemi-L-tartrate, citrate, phosphate, hemi-naphthylene-1,5-disulphonate, hemi-fumarate, sulfate, mesylate, acetate, hemi-oxalate, and oxalate.

22. The method of claim 18, wherein the salt is chosen from the group consisting of hydrochloride pattern A, phosphate pattern A, phosphate pattern B, phosphate pattern C, HBr pattern A, HBr pattern B, HBr pattern C, hemi-L-tartrate pattern A, hemi-meso-tartrate pattern B, hemi-meso-tartrate pattern C, meso-tartrate pattern A, meso-tartrate pattern B, sulfate pattern A, sulfate pattern B, D-tartrate pattern A, D-tartrate pattern B, D-tartrate pattern C, D-tartrate pattern D, D-tartrate pattern E, L-maleate pattern A, maleate pattern A, maleate pattern B, hemi naptheylene-1,5-disulfonate pattern A, hemi naptheylene-1,5-disulfonate pattern B, hemi-oxalate pattern A, hemi-oxalate pattern A′, hemi-fumarate pattern A, hemi-fumarate pattern A′, mesylate pattern A, acetate pattern A, citrate pattern A, fumarate pattern A, and oxalate pattern A.

23. The method of claim 18, wherein the composition is administered in a dose of 10-1000 mg.

24. The method of claim 18, wherein the composition is administered daily.

25. The method of claim 18, wherein the composition is formulated in a continual slow-release formulation.

26. The method of claim 25, wherein said composition is formulated in a transdermal patch.

27. The method of claim 18, wherein said composition is formulated in an intranasal spray.

28. The method of claim 18, wherein said composition is formulated in a liquid dosage form chosen from the group consisting of suspensions, solutions, emulsions, elixirs, tinctures, sprays, syrups, gels, magmas, liniments, lotions, ointments, pastes, drops, and inhalants.

29. The method of claim 18, wherein said composition is formulated in a solid dosage form chosen from the group consisting of capsules, films, lozenge, patch, powder, tablets, pellets, pills, and troches.

30. The composition of claim 1, wherein said acid is hydrochloric acid, and said crystalline form is characterized by an x-ray powder diffraction pattern having peaks expressed as 2θ at about 15.8, about 17.5, about 19.7, about 24.8, and about 24.9.

31. The composition of claim 1, wherein said acid is hydrobromic acid, and said crystalline form is characterized by an x-ray powder diffraction pattern having peaks expressed as 2θ at about 13.9, about 16.3, about 19.8, about 20.5, and about 24.0.

32. The composition of claim 1, wherein said acid is phosphoric acid, and said crystalline form is characterized by an x-ray powder diffraction pattern having peaks expressed as 2θ at about 13.4, about 14.6, about 17.4, about 18.7, and about 22.1.

33. The composition of claim 1, wherein said acid is D-tartaric acid, and said crystalline form is characterized by an x-ray powder diffraction pattern having peaks expressed as 2θ at about 6.0, about 12.0, about 13.3, about 17.9, and about 24.1.

34. The composition of claim 1, wherein said acid is fumaric acid, and said crystalline form is characterized by an x-ray powder diffraction pattern obtained by irradiation with Cu Kα x-rays having peaks expressed as 2θ at about 17.2, about 18.6, about 19.2, about 19.5, and about 21.8.

35. The composition of claim 34, wherein said salt is a hemi-salt.

36. The composition of claim 1, wherein said acid is oxalic acid, and said crystalline form is characterized by an x-ray powder diffraction pattern obtained by irradiation with Cu Kα x-rays having peaks expressed as 2θ at about 15.2, about 16.4, about 16.8, about 19.3, and about 21.3.

37. The corn position of claim 33, wherein said salt is a hemi-salt.

38. The composition of claim 1, wherein said acid is hydrobromic acid, and said crystalline form is characterized by an x-ray powder diffraction pattern obtained by irradiation with Cu Kα x-rays having peaks expressed as 2θ at about 13.9, about 16.2, about 16.9, about 20.5, and about 24.1.

39. The composition of claim 1, wherein said acid is phosphoric acid, and said crystalline form is characterized by an x-ray powder diffraction pattern having peaks expressed as 2θ at about 14.5, about 17.4, about 22.0, about 24.7, and about 24.9.

40. The composition of claim 1, wherein said acid is phosphoric acid, and said crystalline form is characterized by an x-ray powder diffraction pattern having peaks expressed as 2θ at about 12.9, about 13.8, about 17.1, about 26.8, and about 27.8.

41. The composition of claim 1, wherein said acid is D-tartaric acid, and said crystalline form is characterized by an x-ray powder diffraction pattern having peaks expressed as 2θ at about 5.6, about 11.3, about 15.4, about 17.2, and about 17.8.

42. The composition of claim 1, wherein said acid is D-tartaric acid, and said crystalline form is characterized by an x-ray powder diffraction pattern having peaks expressed as 2θ at about 5.1, about 16.3, about 19.3, about 20.4, and about 21.8.

43. The composition of claim 1, wherein said acid is maleic acid, and said crystalline form is characterized by an x-ray powder diffraction pattern having peaks expressed as 2θ at about 14.9, about 18.0, about 25.2, about 25.9, and about 27.9.

44. The composition of claim 1, wherein said acid is malic acid, and said crystalline form is characterized by an x-ray powder diffraction pattern having peaks expressed as 2θ at about 17.8, about 18.1, about 19.3, about 26.5, and about 27.3.

45. The composition of claim 1, wherein said acid is napthylene-1,5-disulfonic acid, and said crystalline form is characterized by an x-ray powder diffraction pattern having peaks expressed as 2θ at about 14.6, about 15.2, about 15.8, about 16.8, and about 22.9.

46. The composition of claim 45, wherein said salt is a hemi-salt.

47. The composition of claim 1, wherein said acid is oxalic acid, and said crystalline form is characterized by an x-ray powder diffraction pattern having peaks expressed as 2θ at about 4.8, about 14.6, about 16.8, about 19.9, and about 21.0.

48. The composition of claim 1, wherein said acid is sulfuric acid, and said crystalline form is characterized by an x-ray powder diffraction pattern having peaks expressed as 2θ at about 14.9, about 17.8, about 21.0, about 21.2, and about 23.8.

49. The composition of claim 1, wherein said acid is methanesulfonic acid, and said crystalline form is characterized by an x-ray powder diffraction pattern having peaks expressed as 2θ at about 16.2, about 17.9, about 18.5, about 21.2, and about 26.9.

50. The composition of claim 1, wherein said acid is acetic acid, and said crystalline form is characterized by an x-ray powder diffraction pattern having peaks expressed as 2θ at about 17.7, about 18.0, about 18.6, about 19.7, and about 20.3.
